SEQUENCE LISTING (5 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 94 aa, Homo sapiens
QASMTASPPGSLRASDWVILNCSFSRPDRPASVHWFRNRGQGRVPVRESPHHHLAESFLFLPQVSPMDSGPWGCILTYRDGFNVSIMYNLTVLG

SEQ ID NO: 3 - protein, 94 aa, Macaca fascicularis
QASMTASPPGSLRTSDWVILNCSFSRPDRPASVHWFRSRGQGRVPVQGSPHHHLAESFLFLPHVGPMDSGLWGCILTYRDGFNVSIMYNLTVLG

SEQ ID NO: 4 - protein, 92 aa, Mus musculus
QASMIASPSGVLKLSDWVLLNCSFSRPDRPVSVHWFQGQNRVPVYNSPRHFLAETFLLLPQVSPLDSGTWGCVLTYRDGFNVSITYNLKVLG

SEQ ID NO: 5 - protein, 92 aa, Rattus norvegicus
QASMIASPPGTLKPSDWVILNCSFSRPDPRVSVHWFQGQSRVPVHNSPRHYLAESFLLLPQVSPLDSGTWGCVLTYRDGFNVSITYNLKVQG

(19) United States
(12) Patent Application Publication
(10) Pub. No.: US 2025/0198990 A1
(43) Pub. Date: Jun. 19, 2025

(54) Methods of Identifying Anti-LAG-3 Agents

(71) Applicants: The Board of Trustees of the Leland Stanford Junior University, Stanford, CA (US); New York University, New York, NY (US)

(72) Inventors: Jack Silberstein, Stanford, CA (US); Jennifer R. Cochran, Stanford, CA (US); Jasper Du, New York, NY (US); Jun Wang, New York, NY (US)

(21) Appl. No.: 18/846,608

(22) PCT Filed: Mar. 15, 2023
(86) PCT No.: PCT/US2023/064469

§ 371 Date: Sep. 12, 2024

Related U.S. Application Data
(60) Provisional application No. 63/319,982, filed on Mar. 15, 2022.

(51) Int. Cl. G01N 33/50; C07K 16/28

(52) U.S. Cl. CPC G01N 33/505; C07K 16/2803; C07K 2317/76; G01N 2500/04; G01N 2500/10

(57) ABSTRACT

Provided are methods of generating cells that produce monoclonal antibodies that specifically bind domain 2 of a human lymphocyte activation gene-3 (LAG-3) polypeptide. The methods comprise immunizing a non-human animal with an immunogen comprising domain 2 of a human LAG-3 polypeptide and isolating monoclonal antibody-producing cells from the non-human animal, wherein the monoclonal antibody-producing cells produce monoclonal antibodies that specifically bind the immunogen. Such methods further comprise screening the monoclonal antibody-producing cells for cells that produce monoclonal antibodies that specifically bind domain 2 of human LAG-3. Also provided are in vitro methods of identifying a polypeptide that specifically binds domain 2 of a human LAG-3 polypeptide, as well as in silico methods of identifying an agent as a candidate human LAG-3 dimerization disrupting agent. Antibodies, polypeptides and agents identified according to the methods of the present disclosure are also provided.

### CROSS-REFERENCE TO RELATED APPLICATIONS

This application claims the benefit of U.S. Provisional Patent Application No. 63/319,982, filed Mar. 15, 2022, which application is incorporated herein by reference in its entirety.

### INTRODUCTION

T cells utilize checkpoint receptors to ensure T cell receptor (TCR) recognition of peptide displayed by major histocompatibility complex (pMHC) leads to functional responses that are not autoreactive. Upon functional engagement of TCR with cognate pMHC, a T cell becomes activated and clonally expands. To prevent overactivation and autoreactivity, activated T cells will upregulate checkpoint receptors to avoid activation against self pMHC. Lymphocyte activation gene-3 (LAG-3) is an immune checkpoint receptor expressed by activated CD4⁺ and CD8⁺ T cells and is known to regulate T cell function.

LAG-3 is a single-pass transmembrane receptor, consisting of four extracellular immunoglobulin-like domains, a transmembrane domain, and an unusual cytoplasmic tail that contains a glutamic acid-proline tandem repeat. Noted for its homology to CD4 in sequence and chromosomal location, LAG-3 is thought to be the result of gene duplication. Like CD4, LAG-3 also binds to MHCII. However, rather than delivering an activating signal through kinase recruitment like CD4, LAG-3 has been shown to disrupt the signaling cascade of T cells. Absent a classical immunoreceptor tyrosine-based inhibition motif (ITIM), LAG-3 is thought to disrupt signaling through its negatively charged cytoplasmic tail. Recent evidence suggests that the LAG-3 cytoplasmic glutamic acid-proline repeat is involved in blocking the association of activating kinases with the cytoplasmic tails of the CD4 and CD8 co-receptors.

Previous reports have suggested that LAG-3 dimerizes through its first, most membrane distal domain (D1), which is also involved in MHCII ligation. Despite the success of anti-LAG-3 antibodies in clinical trials for the treatment of cancer, the mechanism by which LAG-3 functions remains poorly understood.

### SUMMARY

Provided are methods of generating cells that produce monoclonal antibodies that specifically bind domain 2 of a human lymphocyte activation gene-3 (LAG-3) polypeptide. The methods comprise immunizing a non-human animal with an immunogen comprising domain 2 of a human LAG-3 polypeptide and isolating monoclonal antibody-producing cells from the non-human animal, wherein the monoclonal antibody-producing cells produce monoclonal antibodies that specifically bind the immunogen. Such methods further comprise screening the monoclonal antibody-producing cells for cells that produce monoclonal antibodies that specifically bind domain 2 of the human LAG-3 polypeptide. Also provided are in vitro methods of identifying a polypeptide that specifically binds domain 2 of a human LAG-3 polypeptide, as well as in silico methods of identifying an agent as a candidate human LAG-3 dimerization disrupting agent. Antibodies, polypeptides and agents identified according to the methods of the present disclosure, as well methods of using such antibodies, polypeptides and agents, are also provided.

### BRIEF DESCRIPTION OF THE FIGURES

FIG. 1: Crystal structure of LAG-3 dimer.

FIG. 2A-2B: Epitope mapping reveals that the C9B7W anti-mouse LAG-3 antibody binds domain 2 of mouse LAG-3.

FIG. 3A-3B: Electron microscopy reveal that the C9B7W anti-mouse LAG-3 antibody functions by blocking LAG-3 dimerization. 3A: electron micrographs showing LAG-3 alone. 3B: electron micrographs showing disruption of LAG-3 dimerization by treatment of LAG-3 with C9B7W.

FIG. 4A-4B: Data demonstrating that blocking LAG-3 dimerization/signaling and maintaining MHCII binding is superior to knocking out LAG-3.

FIG. 5: FRET data demonstrating that both mouse and human LAG-3 can dimerize.

FIG. 6A-6B: Amino acid residues within the LAG-3 dimerization interface in mouse, rat, human and cynomolgus monkey. The right-most column shows the mouse (FIG. 6A) and human (FIG. 6B) amino acids residues in the opposite chain of the dimer within 5 angstroms of the residues indicated to the left in the same row.

FIG. 7A-7C: Overall structure of the LAG-3 dimer. (A) Negative stain electron microscopy 3D reconstruction map model of LAG-3 dimer and selected reference-free 2D class averages. Four extracellular domains (D1-D4) were labeled on the corresponding positions in both LAG-3 molecules of the dimer map model. (B) Surface representation of the extracellular domain of the LAG-3 dimeric crystal structure. Missing portions of the molecule are indicated with dashed lines. (C) Surface representation of CD4-MHCII-TCR (PDB 3T0E) superimposed onto TCR-CD3 (PDB 6JXR).

FIG. 8A-8C: Structural comparisons of glycosylated mouse LAG-3 with human CD4 and deglycosylated mouse LAG-3 domains 1 and 2. (A) Cartoon structure of the mouse LAG-3 ECD aligned with the human CD4 ECD structure (PDB 1WIO). Angular differences between domain 1 of CD4 and LAG-3 allow additional space for LAG-3's long extra loop (loop 1) in the LAG-3 dimer. (B) Cartoon structure of the deglycosylated mouse LAG-3 domains 1 and 2 (PDB 7TZE) compared to the glycosylated mouse LAG-3 domains 1 and 2. The glycan-protein interaction at the top of the D2 dimer interface brings two monomers together and increased the buried surface area from 440 angstroms in the deglycosylated structure to 511 angstroms in the glycosylated structure. (C) Cartoon structure of the deglycosylated human LAG-3 bound to F7 scFv (PDB 7TZG) compared to the glycosylated mouse LAG-3.

FIG. 9A-9G: Glycosylation and homodimerization at the D2 dimer interface. (A) Ribbon representation of domains 1-3 in the dimeric LAG-3 structure. Glycans are shown in green. (B) SDS-PAGE of mouse LAG-3 extracellular domain expressed and purified from Expi293F cells (Lane 1), Expi293F GnTI-cells (Lane 2), and Expi293F GnTI-cells, followed by EndoHf treatment for 16 hours at 4° C. (Lane 3) or 1 hour at 37° C. (Lane 4). The predicted molecular weights of the LAG-3 extracellular domain and EndoHf are 46.2 kDa and 70 kDa, respectively. The shift in molecular weight between mouse LAG-3 in lanes 1 and 4 represents approximately 10 kDa of N-linked glycans. Asterisk represents the protein that was used for crystal structure determination. (C) Contact residues in the D2 dimer interface are shown. R192 and the negatively charged glycan on N184 can be visualized in the top view. (D) The side view shows an extensive network of hydrophobic interacting residues. (E) Graphical representation of flow-based FRET experiments using two-chain, two-plasmid system with Exip293F cells. (F) Flow-based FRET data from Expi293F cells comparing CFP-YFP fused by a linker to double transfections of mLAG-3^{CFP}/CD80^{YFP}, CD80^{CFP}/CD80^{YFP} (known homodimer), mLAG-3^{CFP}/mLAG-3^{YFP}, and hLAG-3^{CFP}/hLAG-3^{YFP}. (G) Flow-FRET data showing that hydrophobic-to-charged mutation W180D significantly reduces LAG-3 dimerization. All statistics were determined by Student's t-test with P values noted in the figure.

FIG. 10A-10B: LAG-3 domain 2 interface is highly hydrophobic. (A) Surface representation showing the hydrophobic patch of domain 2 that mediates LAG-3 dimer formation. (B) Sequence alignment of domain 2 from humans, cynomolgus macaques, mice, and rats, colored by percentage identity (from top to bottom SEQ ID NOs: 2-5). Arrows identify fully conserved hydrophobic and N-linked glycan residues that mediate LAG-3 dimerization.

FIG. 11A-11H: Dimerization has a role in LAG-3 function. LAG-3 variants were stably transfected into 3A9 cells and tested for their ability to bind (A) pMHCII tetramer, (B) FGL1 dimer, and (C) an antibody against D3 (410C9). (D) Mutations in the pMHCII binding site or dimerization interface of LAG-3 prevent it from functioning properly when expressed on the surface of 3A9 cells co-cultured with LK35.2 B cells. (E) Representative histograms of DO11.10 cells stably expressing LAG-3 variants. (F-H) LAG-3 variant-expressing DO11.10 cells were tested in the same assays as A-D. All statistics were determined by Student's t-test with P values noted in the figure.

FIG. 12A-12B: Yeast surface display of mouse LAG-3 domains 1 and 2 for fine epitope mapping. (A) Domains 1 and 2 of mouse LAG-3 were displayed on the surface of yeast for fine epitope mapping. (B) Fine epitope mapping of TKB58 reveals a binding epitope at the tip of D1, similar to that of M8-4-6. Binding epitope is shown on one chain for clarity; however, the epitope is present in the opposite chain as well.

FIG. 13A-13B: Fine epitope mapping reveals functional epitopes for LAG-3 antibodies. Domains 1 and 2 of mouse LAG-3 were displayed on the surface of yeast and incubated with the corresponding antibodies. (A) Fine epitope mapping of C9B7W reveals a binding epitope at the dimer interface of LAG-3. (B) Fine epitope mapping of M8-4-6 reveals a binding epitope at the tip of D1.

FIG. 14A-14E: Dimer disrupting antibodies block LAG-3 function. Negative stain electron microscopy showing selected 2D class averages and the 3D map model of (A) the C9B7W Fab/LAG-3, (B) the M8-4-6 Fab/LAG-3, and (C) the 410C9 Fab/LAG-3 complexes. The densities corresponding to the Fabs and LAG-3 (blue) are indicated by arrows in the class averages. (D) Selected 2D class averages of the M7-6-9-5 Fab/LAG-3 complex. The LAG-3 dimer is visualized, with two Fabs (purple arrow) bound to each dimer. (E) Co-culture of 293T cells stably expressing pMHCII and 3A9 cells stably expressing a NFkB-GFP reporter and a LAG-3 CAR. C9B7W (anti-D2), M8-4-6 (anti-D1), or 410C9 (anti-D3) are capable of potently blocking LAG-3-mediated CAR signaling. All statistics were determined by Student's t-test with P values noted in the figure.

FIG. 15A-15F: LAG-3 CAR assay with various LAG-3 ligands. (A) 293T cells stably expressing LAG-3 ligands were co-cultured with 3A9 cells stably expressing a NFkB-GFP reporter and a LAG-3 CAR. The LAG-3 CAR consists of the mouse LAG-3 ECD, followed by CD28/41BB/CD3z ICD. Upon ligand engagement by this LAG-3 CAR, the 3A9 cells become activated, which leads to NFkB-dependent GFP expression. This assay was also used for generating the I-A^{k}-HEL data presented in FIG. 14E. In this figure. LAG-3 ligands HLA-DR1 HA and transmembrane-fused mFGL1 were used to test antibody blocking capabilities.

### DETAILED DESCRIPTION

Before the methods and agents of the present disclosure are described in greater detail, it is to be understood that the methods and agents are not limited to particular embodiments described, as such may, of course, vary. It is also to be understood that the terminology used herein is for the purpose of describing particular embodiments only, and is not intended to be limiting, since the scope of the methods and agents will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and agents. The upper and lower limits of these smaller ranges may independently be included in the smaller ranges and are also encompassed within the methods and agents, subject to any specifically excluded limit in the stated range. Where the stated range includes one or both of the limits, ranges excluding either or both of those included limits are also included in the methods and agents.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and agents belong. Although any methods and agents similar or equivalent to those described herein can also be used in the practice or testing of the methods and agents, representative illustrative methods and agents are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and agents are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and agents, which are, for clarity, described in the context of separate embodiments, may also be provided in combination in a single embodiment. Conversely, various features of the methods and agents, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and agents and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

In Vivo Methods of Generating Cells that Produce Monoclonal Antibodies Specific for Domain 2 of Human LAG-3

The present disclosure provides methods of generating cells that produce monoclonal antibodies that specifically bind domain 2 of a human lymphocyte activation gene-3 (LAG-3) polypeptide. The methods comprise immunizing a non-human animal with an immunogen comprising or encoding domain 2 of a human LAG-3 polypeptide and isolating monoclonal antibody-producing cells from the non-human animal, wherein the monoclonal antibody-producing cells produce monoclonal antibodies that specifically bind the immunogen. Such methods further comprise screening the monoclonal antibody-producing cells for cells that produce monoclonal antibodies that specifically bind domain 2 of the human LAG-3 polypeptide.

These and the other methods described below (e.g., methods of identifying polypeptides that specifically bind domain 2 of human LAG-3, and methods of identifying agents as candidate human LAG-3 dimerization disrupting agents) are based in part on the inventors' unexpected findings that: based on the crystal structure of the LAG-3 dimer, dimerization of LAG-3 is via a domain 2 dimerization interface of LAG-3; the epitope of an anti-LAG-3 antibody (C9B7W) with desirable pharmacologic properties maps to the domain 2 dimerization interface of LAG-3; the anti-LAG-3 antibody (C9B7W) functions by disrupting LAG-3 dimerization; and targeting LAG-3 via this mechanism (disrupting LAG-3 dimerization/signaling) is superior to knocking out LAG-3 or targeting domain 1 of LAG-3, where current anti-LAG-3 therapeutics typically target domain 1 and lack satisfactory clinical efficacy, e.g., in treating cancer.

Also provided by the present disclosure are methods of generating cells that produce monoclonal antibodies that specifically bind an extracellular domain of a human LAG-3 polypeptide. Such methods comprise immunizing a non-human animal with an immunogen comprising one or more extracellular domains of a human LAG-3 polypeptide, and isolating monoclonal antibody-producing cells from the non-human animal, wherein the monoclonal antibody-producing cells produce monoclonal antibodies that specifically bind the immunogen. Such methods further comprise screening the monoclonal antibody-producing cells for cells that produce monoclonal antibodies that disrupt dimerization of the human LAG-3 polypeptide. In certain embodiments, the cells produce monoclonal antibodies that specifically bind domain 2 of the human LAG-3 polypeptide. According to some embodiments, the cells produce monoclonal antibodies that specifically bind a domain of the human LAG-3 polypeptide other than domain 2 (e.g., domain 1 or domain 3), where binding of the monoclonal antibodies to the domain other than domain 2 disrupts dimerization of the human LAG-3 polypeptide, e.g., via an allosteric effect on the domain 2 dimerization interface. Immunogens, non-human animals, dimerization screening approaches, etc. that find use in practicing the methods are described elsewhere herein.

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In some embodiments, the antibody is selected from an IgG, Fv, single chain antibody, scFv, Fab, F(ab′)₂, or Fab′. The antibodies may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, yeast or phage clone, or produced via a cell-free expression system, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, yeast display technologies, phage display technologies, ribosome display technologies, DNA display technologies, and the like. For example, monoclonal antibodies may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of and/or selective for the antigen in the presence of a heterogeneous population of antigens (e.g., proteins and other biologics, e.g., in a sample or in vivo). Thus, under designated assay (e.g., immunoassay) conditions, the specified polypeptides bind to a particular antigen and do not bind in a significant amount to other antigens present in the sample. Specific binding to an antigen under such conditions may require a polypeptide that is selected for its specificity for a particular antigen. For example, a polypeptide (e.g., an antibody) can specifically bind to a human LAG-3 antigen comprising domain 2 of LAG-3, and does not exhibit comparable binding (e.g., does not exhibit detectable binding) to other proteins present in a sample.

In some embodiments, a polypeptide (e.g., antibody) of the present disclosure “specifically binds” a human LAG-3 antigen comprising domain 2 of LAG-3 if it binds to or associates with the human LAG-3 antigen comprising domain 2 of LAG-3 with an affinity or Kₐ (that is, an equilibrium association constant of a particular binding interaction with units of 1/M) of, for example, greater than or equal to about 10⁵ M⁻¹. In certain embodiments, the antibody binds to the human LAG-3 antigen comprising domain 2 of LAG-3 with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the polypeptide binds to the human LAG-3 antigen comprising domain 2 of LAG-3 with a Kₐ of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the polypeptide for the human LAG-3 antigen comprising domain 2 of LAG-3 can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer): by radioimmunoassay; or the like.

An “epitope” is a site on an antigen to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology. Vol. 66, Glenn E. Morris, Ed (1996). Several commercial laboratories offer epitope mapping services. Epitopes bound by an antibody immunoreactive with a LAG-3 antigen can reside, e.g., on the surface of the LAG-3 antigen, so that such epitopes are considered LAG-3-surface accessible, solvent accessible, and/or LAG-3-surface exposed.

As summarized above, the methods of generating cells that produce monoclonal antibodies that specifically bind domain 2 of a human LAG-3 polypeptide comprise immunizing a non-human animal with an immunogen comprising domain 2 of a human LAG-3 polypeptide. In certain embodiments, the immunogen consists of the extracellular domain (ECD) of a human LAG-3 polypeptide or a fragment thereof comprising domain 2. In this context, “consists of” means that the immunogen does not include domains of LAG-3 other than the ECD, and the immunogen may be a fragment of the ECD, e.g., the immunogen may include an N-terminal and/or C-terminal truncation as compared to the full-length wild-type ECD.

The amino acid sequence of a human LAG-3 polypeptide is provided in Table 1 below. Italicized amino acids 1-28 are a signal sequence. Italicized amino acids 430-525 are C-terminal to the extracellular domain. Non-italicized amino acids constitute extracellular domains 1-4 indicated by alternating underlining, where domain 1 is underlined (amino acids 29-167), domain 2 not underlined (amino acids 168-261), domain 3 is underlined (amino acids 262-349) and domain 4 is not underlined (amino acids 350-429)

TABLE 1
Amino acid sequence of a human LAG-3 polypeptide

| | |
|---|---|
| Human LAG-3 | MWEAQFLGLLFLQPLWVAPVKPLQPGAEVPVVWAQEGAPAQLPCSPTIP |
| polypeptide | LQDLSLLRRAGVTWQHQPDSGPPAAAPGHPLAPGPHPAAPSSWGPRPR |
| (SEQ ID NO: 1) | RYTVLSVGPGGLRSGRLPLQPRVQLDERGRQRGDFSLWLRPARRADAG |
| | EYRAAVHLRDRALSCRLRLRLGQASMTASPPGSLRASDWVILNCSFSRPD |
| | RPASVHWFRNRGQGRVPVRESPHHHLAESFLFLPQVSPMDSGPWGCILT |
| | YRDGFNVSIMYNLTVLGLEPPTPLTVYAGAGSRVGLPCRLPAGVGTRSFL |
| | TAKWTPPGGGPDLLVTGDNGDFTLRLEDVSQAQAGTYTCHIHLQEQQLN |
| | ATVTLAIITVTPKSFGSPGSLGKLLCEVTPVSGQERFVWSSLDTPSQRSFS |
| | GPWLEAQEAQLLSQPWQCQLYQGERLLGAAVYFTELSSPGAQRSGRAP |
| | GALPAGHLLLFLILGVLSLLLLVTGAFGFHLWRRQWRPRRFSALEQGIHPP |
| | QAQSKIEELEQEPEPEPEPEPEPEPEPEPEQL |

According to some embodiments, the immunogen consists of domains 1 and 2 of a human LAG-3 polypeptide or a fragment thereof comprising domain 2. In this context, “consists of” means that the immunogen does not include domains of LAG-3 other than domains 1 and 2, and the immunogen may be a fragment of such an immunogen, e.g., the immunogen may include an N-terminal and/or C-terminal truncation as compared to the full-length wild-type combined sequence of domains 1 and 2.

In certain embodiments, the immunizing comprises administering the immunogen to the non-human animal. According to some embodiments, the immunizing comprises administering a nucleic acid (e.g., mRNA or DNA) encoding the immunogen to the non-human animal, wherein the nucleic acid is configured to express the immunogen upon administration to the non-human animal. In certain embodiments, the immunizing comprises administering cells that express the immunogen to the non-human animal, wherein the cells secrete the immunogen and/or display the immunogen on their surface upon administration to the non-human animal.

In certain embodiments, the screening comprises identifying, among the monoclonal antibody-producing cells, cells that produce monoclonal antibodies that bind to a human LAG-3 polypeptide consisting of domain 2 or a fragment thereof. According to some embodiments, the screening comprises performing single cell sequencing on the monoclonal antibody-producing cells, determining and analyzing the sequences of the antibodies produced by such single cells, and identifying candidate human LAG-3 domain 2-binding antibodies based on the determined and analyzed antibody sequences. Alternatively or additionally, the screening may comprise a binding assay for determining which cells produce monoclonal antibodies that bind to a human LAG-3 fragment consisting of domain 2 or a fragment thereof, e.g., a human LAG-3 fragment consisting of amino acids 175 to 230 of the human LAG-3 polypeptide set forth in SEQ ID NO:1 or a fragment thereof comprising amino acids I186, E215 and H220, wherein numbering is according to SEQ ID NO:1. Alternatively, or additionally, the screening may comprise a negative selection procedure to eliminate cells that produce monoclonal antibodies that bind to a domain of the human LAG-3 polypeptide other than domain 2. Such a negative selection procedure may comprise, e.g., a binding assay for determining which cells produce monoclonal antibodies that bind to a human LAG-3 fragment which does not comprise domain 2, and eliminating those cells. According to some embodiments, the screening comprises a negative selection procedure to eliminate cells that produce monoclonal antibodies that bind to a region of the human LAG-3 polypeptide other than a region within amino acids 175 to 230 of the human LAG-3 polypeptide set forth in SEQ ID NO:1. For example, in certain embodiments, a negative selection procedure comprises a binding assay for determining which cells produce monoclonal antibodies that retain binding to a human LAG-3 fragment that comprises domain 2 or a portion thereof comprising an amino acid substitution (e.g., alanine substitution) at one or more of I186, E215 and H220, which amino acids have been determined by the inventors to play a role in human LAG-3 dimerization. Cells that produce monoclonal antibodies that bind to such an amino acid substituted domain 2 of human LAG-3 may then be identified as cells which are not likely to disrupt dimerization of human LAG-3, and may be eliminated on that basis. Screening assays for positively or negatively selecting for cells and antibodies based on antibody binding properties are known and include enzyme-linked immunosorbent assay (ELISA), flow cytometry (e.g., fluorescence-activated cell sorting (FACS)), the gel encapsulated microdroplet (GEM) assay (Crystal Bioscience/Ligand Pharmaceuticals), the xPloration™ platform (Ligand Pharmaceuticals), the high-throughput single-cell screening platform of AbCellera Biologics Inc., the Beacon® high-throughput single-cell screening platform of Berkeley Lights, Inc., and the like.

In certain embodiments, the non-human animal is a mouse, a rat, a chicken, a cow, a goat, a llama/alpaca, a shark, or a rabbit. According to some embodiments, the non-human animal comprises a replacement of one or more endogenous non-human animal immunoglobulin (Ig) genes with human Ig genes. Such genetically modified non-human animals are available and include: those among the OmniAb® platform (Ligand Pharmaceuticals, Inc.) which includes the OmniMouse®, OmniRat, and OmniChicken animals; XenoMouse® (Amgen); HuMAbMouse (Bristol Myers Squibb); KyMouse™ (Kymab); Velocimmune® mouse (Regeneron Pharmaceuticals Inc.); H2L2 mouse and HCAb mouse (Harbour Antibodies BV); Trianni Mouse® (Trianni Inc.); ATX-Gx™ mice (Alloy Therapeutics); the AlivaMab® mouse (AlivaMab); and the like. Accordingly, in certain embodiments, the monoclonal antibody-producing cells produce monoclonal antibodies comprising one or more human antibody domains (i.e., human/non-human animal chimeric antibodies) that specifically bind the immunogen, and in some instances, fully human monoclonal antibodies that specifically bind the immunogen.

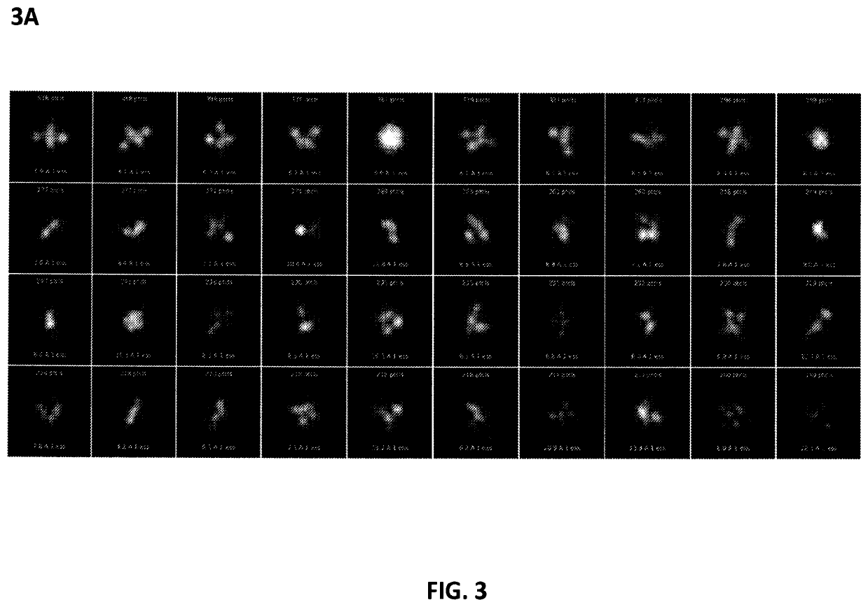

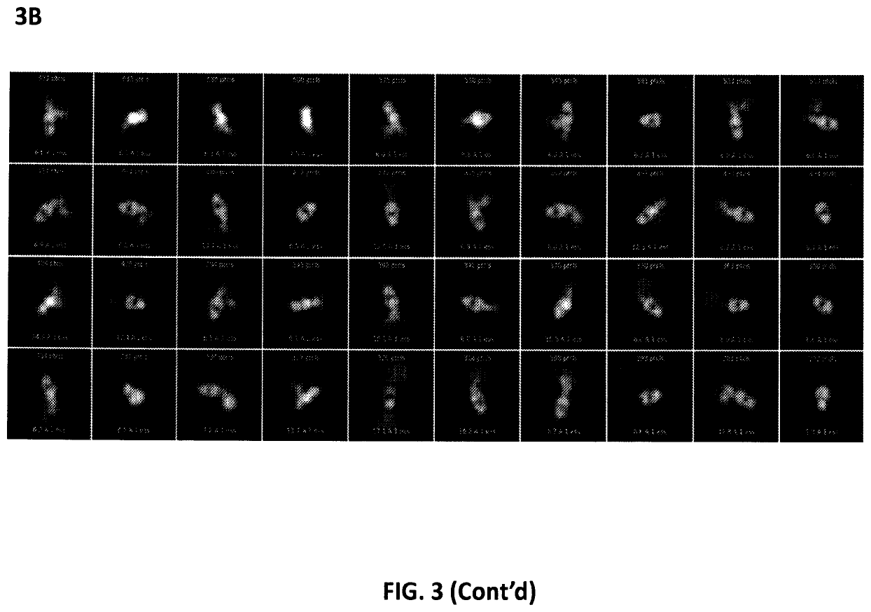

According to some embodiments, the method further comprises screening monoclonal antibodies identified as specifically binding domain 2 of the human LAG-3 polypeptide for the ability to disrupt human LAG-3 dimerization. The screening may comprise, e.g., combining human LAG-3 monomers and/or dimers with monoclonal antibodies produced by cells generated via the methods of the present disclosure (or monoclonal antibodies comprising the six CDRs of such antibodies) and assessing for the presence or absence of human LAG-3 dimers, e.g., by sodium dodecyl sulphate-polyacrylamide gel electrophoresis (SDS-PAGE), size exclusion chromatography, analytical ultracentrifugation, electron microscopy (see, e.g., Example 2 and FIG. 3 herein), or the like.

A variety of suitable approaches for preparing immunogens, nucleic acids that encode a desired immunogen (e.g., when a nucleic acid encoding the immunogen is administered to the non-human animal), cells that express a desired immunogen (e.g., when cells expressing the immunogen are administered to the non-human animal), immunizing non-human animals, isolating monoclonal antibody-producing cells from non-human animals, etc. are known by those of ordinary skill in the art. When the immunizing comprises administering the immunogen to the non-human animal, an appropriate immunogenic preparation can contain, e.g., a recombinantly-expressed immunogen comprising domain 2 of the human LAG-3 polypeptide or a chemically-synthesized immunogen comprising domain 2 of the human LAG-3 polypeptide. Immunogenicity of the immunogen can be increased by fusion or conjugation to a hapten such as keyhole limpet hemocyanin (KLH) or ovalbumin (OVA). One can also combine the immunogen with a conventional adjuvant such as Freund's complete or incomplete adjuvant to increase the non-human animal's immune reaction to the immunogen. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds.

Immune responses may be described as either “primary” or “secondary” immune responses. A primary immune response, which is also described as a “protective” immune response, refers to an immune response produced in an individual as a result of some initial exposure (e.g., the initial “immunization”) to a particular immunogen, e.g., an immunogen comprising domain 2 of the human LAG-3 polypeptide. In some embodiments, the immunization can occur as a result of vaccinating the non-human animal with a vaccine containing the immunogen. For example, the vaccine can be a human LAG-3 vaccine comprising one or more species of LAG-3 polypeptides comprising domain 2. A primary immune response can become weakened or attenuated over time and can even disappear or at least become so attenuated that it cannot be detected. Accordingly, the methods may comprise a “secondary” immune response, which is also described here as a “memory immune response.” The term secondary immune response refers to an immune response elicited in an individual after a primary immune response has already been produced. Thus, a secondary immune response can be elicited, e.g., to enhance an existing immune response that has become weakened or attenuated, or to recreate a previous immune response that has either disappeared or can no longer be detected.

According to the methods of the present disclosure, monoclonal antibodies may be produced using any technique known in the art. e.g., by immortalizing spleen cells harvested from the non-human animal after completion of the immunization schedule. The spleen cells can be immortalized using any technique known in the art, e.g., by fusing them with myeloma cells to produce hybridomas. See, for example, Antibodies; Harlow and Lane. Cold Spring Harbor Laboratory Press, 1st Edition, e.g., 2nd Edition, e.g., from 2014. Myeloma cells for use in hybridoma-producing fusion procedures typically are non-antibody-producing, have high fusion efficiency, and enzyme deficiencies that render them incapable of growing in certain selective media, which support the growth of only the desired fused cells (hybridomas). Examples of suitable cell lines for use in fusions with mouse cells include, but are not limited to, Sp-20, P3-X63/Ag8, P3-X63-Ag8.653, FO, NSO/U, MPC-I I, MPC11-X45-GTG 1.7 and S194/5XXO Bul. Examples of suitable cell lines used for fusions with rat cells include, but are not limited to, R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced. Such cell lines may be produced by immunizing an animal (e.g., a mouse, a rat, a chicken, a cow, goat, a llama/alpaca, a shark, or a rabbit-including transgenic versions thereof having human immunoglobulin sequences as described above) with the immunogen comprising domain 2 of the human LAG-3 polypeptide (or nucleic acids encoding the immunogen, or cells expressing the immunogen); harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces monoclonal antibodies that specifically bind domain 2 of the human LAG-3 polypeptide. Another useful method for producing monoclonal antibodies is the SLAM method described in Babcook et al. (1996) Proc. Natl. Acad. Sci. 93:7843-7848. In some instances, B cells of the immunized non-human animal are screened directly, i.e., without producing hybridomas prior to the screening.

Accordingly, the present disclosure also provides cells that produce monoclonal antibodies that specifically bind domain 2 of a human LAG3 polypeptide obtained according to the methods of the present disclosure. Also provided by the present disclosure are monoclonal antibodies produced by such cells. Monoclonal antibodies secreted by such cells (e.g., a hybridoma cell line) can be purified using any technique known in the art, such as protein A-Sepharose, hydroxyapatite chromatography, gel electrophoresis, dialysis, affinity chromatography, or the like. Hybridoma supernatants or monoclonal antibodies may be further screened to identify monoclonal antibodies with particular properties, such as the ability to disrupt human LAG-3 dimerization, disrupt human LAG-3 signaling, and/or the like.

In some embodiments, the monoclonal antibodies of the present disclosure are chimeric or humanized antibodies (which includes antigen-binding fragments thereof) based upon the CDR and variable region sequences of the antibodies generated/identified according to the methods herein. A chimeric antibody is an antibody composed of protein segments from different antibodies that are covalently joined to produce functional immunoglobulin light or heavy chains or binding fragments thereof.

Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567 and Morrison et al., 1985, Proc. Natl. Acad. Sci. USA 81:6851-6855, both of which are hereby incorporated by reference in their entireties.

Generally, the goal of making a chimeric antibody is to create a chimera in which the number of amino acids from the intended species is maximized. One example is the “CDR-grafted” antibody, in which the antibody comprises one or more CDRs from a particular species or belonging to a particular antibody class or subclass, while the remainder of the antibody chain(s) is/are identical with or homologous to a corresponding sequence in antibodies derived from another species or belonging to another antibody class or subclass. CDR grafting is described, for example, in U.S. Pat. Nos. 6,180,370, 5,693,762, 5,693,761, 5,585,089, and 5,530,101. For use in humans, the variable region or selected CDRs from a rodent or rabbit antibody often are grafted into a human antibody, replacing the naturally-occurring variable regions or CDRs of the human antibody.

One useful type of chimeric antibody is a “humanized” antibody. Generally, a humanized antibody is produced from a monoclonal antibody raised initially in a non-human animal, such as a mouse, a rat, a chicken, a cow, a goat, a llama/alpaca, a shark, or a rabbit. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a mouse, a rat, a chicken, a cow, a goat, a llama/alpaca, a shark, or a rabbit variable region for the corresponding regions of a human antibody (see, e.g., U.S. Pat. Nos. 5,585,089, and 5,693,762; Jones et al., 1986, Nature 321:522-525; Riechmann et al., 1988, Nature 332:323-27; and Verhoeyen et al., 1988, Science 239:1534-1536).

In some embodiments, the CDRs of the light and heavy chain variable regions of a monoclonal antibody of the present disclosure are grafted to framework regions (FRs) from antibodies from the same, or a different, phylogenetic species. For example, the CDRs of the heavy and light chain variable regions can be grafted to consensus human FRs. To create consensus human FRs, FRs from several human heavy chain or light chain amino acid sequences may be aligned to identify a consensus amino acid sequence. Alternatively, the grafted variable regions from the one heavy or light chain may be used with a constant region that is different from the constant region of that particular heavy or light chain as disclosed herein. In other embodiments, the grafted variable regions are part of a single chain Fv antibody.

In certain embodiments, the monoclonal antibodies of the present disclosure are human chimeric or fully human antibodies. Human chimeric or fully human antibodies that specifically bind to domain 2 of human LAG-3 can be generated using the immunogens as described elsewhere herein. A “fully human antibody” is an antibody that comprises variable and constant regions derived from or indicative of human germ line immunoglobulin sequences. One specific means provided for implementing the production of human chimeric or fully human antibodies is the “humanization” of the non-human animal humoral immune system. Introduction of human immunoglobulin (Ig) loci into non-human animals in which the endogenous Ig genes have been inactivated is one means of producing human chimeric or fully human antibodies in mice, rats, chickens, etc. that can be immunized with any desirable immunogen. Using human chimeric or fully human antibodies can minimize the immunogenic and allergic responses that can sometimes be caused by administering non-human animal-derived monoclonal antibodies to humans as therapeutic agents.

Once cells producing antibodies that specifically bind to domain 2 of human LAG-3 have been generated according to the methods of the present disclosure, the specific antibody genes may be cloned by isolating and amplifying DNA or mRNA therefrom according to standard procedures as described herein. The antibodies produced therefrom may be sequenced and the CDRs identified. In some embodiments, the DNA coding for the CDRs may be manipulated as described herein to generate other antibodies that specifically bind domain 2 of human LAG-3, e.g., an IgG, a Fab, a F(ab′)₂, a F(ab′), a single chain antibody (e.g., an scFv), a bispecific antibody, a multi-specific antibody, a pH-selective antibody, a protease-activated antibody, and/or the like. Accordingly, the present disclosure also provides nucleic acids that encode any of the monoclonal antibodies of the present disclosure. Such nucleic acids may be operably linked to one or more expression control elements (e.g., promoters, enhancers, and/or the like). As such, also provided are expression vectors comprising the nucleic acids of the present disclosure. Recombinant host cells comprising the nucleic acids or expression vectors of the present disclosure are also provided.

### In Vitro Polypeptide Identification Methods

As summarized above, the present disclosure also provides methods of identifying a polypeptide that specifically binds domain 2 of a human LAG-3 polypeptide. The methods comprise contacting a plurality of human LAG-3 polypeptides with a polypeptide library, wherein each of the plurality of human LAG-3 polypeptides comprise domain 2 of human LAG-3. Such methods further comprise identifying polypeptides of the polypeptide library that specifically bind to human LAG-3 polypeptides of the plurality of human LAG-3 polypeptides, and screening the polypeptides that bind to the human LAG-3 polypeptides for a polypeptide that specifically binds domain 2 of human LAG-3.

Also provided by the present disclosure are methods of identifying a polypeptide that specifically binds the extracellular domain (ECD) of a human LAG-3 polypeptide. Such methods comprise contacting a plurality of human LAG-3 polypeptides with a polypeptide library, wherein each of the plurality of human LAG-3 polypeptides comprise one or more extracellular domains of human LAG-3. Such methods further comprise identifying polypeptides of the polypeptide library that specifically bind to human LAG-3 polypeptides of the plurality of human LAG-3 polypeptides, and screening the polypeptides that bind to the human LAG-3 polypeptides for polypeptides that disrupt dimerization of human LAG-3. In certain embodiments, the polypeptides specifically bind domain 2 of the human LAG-3 polypeptide. According to some embodiments, the polypeptides specifically bind a domain of the human LAG-3 polypeptide other than domain 2 (e.g., domain 1 or domain 3), where binding of the polypeptides to the domain other than domain 2 disrupts dimerization of the human LAG-3 polypeptide, e.g., via an allosteric effect on the domain 2 dimerization interface. Polypeptide libraries, in vitro display formats, dimerization screening approaches, etc. that find use in practicing such methods are described hereinbelow.

According to some embodiments, the human LAG-3 polypeptides of the plurality are displayed on the surface of cells. Suitable cells include, but are not limited to, mammalian cells, yeast cells, and the like. In other embodiments, the human LAG-3 polypeptides of the plurality are displayed on one or more solid supports. As used herein, a “solid support” is an insoluble material to which reagents or material can be attached so that they can be readily separated from the original solution. In certain embodiments, the human LAG-3 polypeptides of the plurality are displayed on the surface of one or more wells. The one or more wells may be one or more wells of a plate, a microfluidic device, or the like. According to some embodiments, the one or more solid supports comprise particulate solid supports. By “particulate solid supports” is meant a collection of solid supports having an average greatest dimension of 1000 micrometers (μm) or less. In some embodiments, the collection of solid supports has an average greatest dimension of 750 μm or less, 500 μm or less, 250 μm or less, 100 μm or less, 1 μm or less, 0.75 μm or less, 0.50 μm or less, 0.25 μm or less, or 0.1 μm or less. In certain embodiments, the particulate solid supports have an average greatest dimension of from about 0.50 μm to about 500 μm, e.g., from about 0.75 μm to about 250 μm. In certain embodiments, the particulate solid supports are beads. As used herein, the term “bead” refers to a small mass that is generally spherical or spheroid in shape. According to some embodiments, a bead as used herein has an average diameter of from about 0.50 μm to about 500 μm, e.g., from about 0.75 μm to about 250 μm.

In certain embodiments, the human LAG-3 polypeptides of the plurality are complexed with ribosome-mRNA (RM) complexes during the contacting, and wherein the mRNAs encode the human LAG-3 polypeptides. For example, a ribosome display methodology for identifying a polypeptide that specifically binds domain 2 of a human LAG-3 polypeptide may be employed. Ribosome display is a completely cell-free system for the in vitro selection of proteins and peptides from large libraries. As it is completely performed in vitro, it circumvents many drawbacks of in vivo systems. In ribosome display, an individual nascent protein couples to its corresponding mRNA through the formation of stable protein-ribosome-mRNA (PRM) complexes, and this complex serves as the physical link between genotype and phenotype. mRNAs are isolated from target-binding complexes, reverse transcribed and amplified as DNA for further manipulation and protein expression. The PRM complex is very stable. The integrity of PRM can be maintained for several days under appropriate conditions, which provide a good way for stringent selections. Besides, ribosome display permits the simultaneous isolation of a functional nascent protein, through affinity for a ligand, together with the encoding mRNA. Ribosome display is described, e.g., in Zahnd et al. (2007) Nat Methods 4, 269-279.

According to some embodiments, the human LAG-3 polypeptides of the plurality are complexed with DNAs during the contacting, and wherein the DNAs encode the human LAG-3 polypeptides. For example, a DNA display methodology for identifying a polypeptide that specifically binds domain 2 of a human LAG-3 polypeptide may be employed. A non-limiting examples of DNA display methodologies which may be employed according to the methods of the present disclosure are described in Yonezawa et al. (2003) Nucleic Acids Res. 31(19):e118; and Bertschinger & Neri (2004) Protein Engineering, Design and Selection 17(9):699-707.

In certain embodiments, the human LAG-3 polypeptides of the plurality are soluble during the contacting step. By “soluble” in this context means the human LAG-3 polypeptides are untethered during the contacting step, e.g., not displayed on a surface (e.g., bead), cell, phage or the like during the contacting step.

According to some embodiments, the polypeptides of the polypeptide library are displayed on the surface of phage during the contacting. For example, a phage display methodology for identifying a polypeptide that specifically binds domain 2 of a human LAG-3 polypeptide may be employed. Phage display technology is based on a direct linkage between phage phenotype and its encapsulated genotype, which leads to presentation of molecule libraries on the phage surface. The gene encoding the displayed molecule is packed within the same virion as a single-strained DNA (ssDNA) and the displayed peptides or proteins are expressed in fusion with phage coat protein. Phage display is utilized in studying protein-ligand interactions, receptor binding sites and in improving or modifying the affinity of proteins for their binding partners. Generating polypeptides and improving their affinity, cloning antibodies from unstable hybridoma cells and identifying epitopes, mimotopes and functional or accessible sites from antigens are also important utilities of this technology. M13 is one of the filamentous bacteriophages (Ff) of Escherichia coli (E. coli), and one of the most widely used phages for antibody phage display. Filamentous bacteriophages only infect E. coli strains through an interaction between the expressed F pilus on the surface of hosts, and a phage coat protein. M13 is a flexible cylindrical-shaped virus particle containing a circular single-stranded DNA genome (6,407-base) consisting of nine genes encoding for five coat proteins (pIII, pVIII, pVI, pVII, and pIX), and six assembly and replication proteins. Most major phage display systems are based on pIII-antibody fusion proteins, due to pIII structural flexibility and its ability to display large proteins without losing its function. The discovery of smaller recombinant antibody formats, such as variable domain [Fv; variable regions of the heavy (V_{H}) or light chain (V_{L})], single-chain variable domain (scFv), diabodies (bivalent scFvs), heavy-domain camelid and shark antibody fragments (VHHs, nanobodies), and fragment antigen binding (Fab), has helped to advance antibody phage display technology. These smaller fragments are more amenable to expression in bacteria compared to full antibodies, which require assembly of four polypeptide chains and extensive disulfide bond formation. For instance, creating a combinatorial scFv library on the surface of M13 filamentous phage has been achieved through combining populations of V_{H} and V_{L}-domains, which are joined by a flexible, protease resistance glycine-serine linker (Gly₄Ser)₃, into a single DNA sequence. These antibody sequences may then be introduced and cloned as a gene fusion with the bacteriophage pIII gene under the control of a weak promoter in a phagemid vector; a plasmid that carries an antibiotic resistance gene, bacterial and phage origins of replication. When purified antigens are available, they can be presented to a phage antibody library by immobilization on solid surfaces, such as nitrocellulose membranes, polystyrene tubes or plates, magnetic beads or column matrices. The use of blocking agents, such bovine serum albumin (BSA), triton/tween, milk or casein can block the remaining sites present on the solid surface to prevent non-specific phage binding to the surface After the phage library is exposed to the immobilized antigens, unbound phages are usually washed away. Such a washing step is critical to remove non-specific binders, and to allow for some control over binding properties by manipulating the wash buffer and stringency of washing. For example, long wash times can be incorporated to ensure only clones with slow dissociation rates are selected. Detergents are usually included in wash buffers, but they can also be altered for factors, such as pH and salt concentration. The washing steps may be gradually increased with every round of biopanning to increase the stringency in order to isolate higher affinity phage clones. Further details regarding phage display are described, e.g., in Bazan et al. (2012) Hum Vaccin Immunother. 8(12):1817-1828; Omidfar & Daneshpour (2015) Expert Opin Drug Discov. 10(6):651-69; Alfaleh et al. (2020) Front. Immunol. doi.org/10.3389/fimmu.2020.01986; Schofield D J, et al. (2007) Genome Biol. 8: R254; and elsewhere. Non-limiting examples of phage display platforms and libraries that may be employed according to the methods of the present disclosure include the SuperHuman™ antibody library (Charles River Laboratories), a phage display library from Twist Bioscience, a phage display library from Specifica Inc., and/or the like.

In certain embodiments, the polypeptides of the polypeptide library are displayed on the surface of cells during the contacting. According to some embodiments, the cells may be mammalian cells. In other embodiments, the cells are yeast cells. For example, a yeast display methodology for identifying a polypeptide that specifically binds domain 2 of a human LAG-3 polypeptide may be employed. The expression of recombinant proteins incorporated into the cell wall of Saccharomyces cerevisiae, termed yeast surface display (YSD), offers selected advantages relative to other display technologies, most notably eukaryotic expression of the heterologous target protein, making it a useful tool for display and engineering of many proteins that are difficult to produce in other display formats. The a-agglutinin system developed by Wittrup et al. uses Aga2p as the display fusion partner. A disulfide linkage between Aga1p, a GPI/β-1,6-glucan-anchored protein, and Aga2p anchors the protein to the cell wall. Thus, co-expression of Aga1p with an Aga2p fusion leads to cell wall-anchored protein on the surface of yeast via disulfide bonding. The Pir (proteins with internal repeats) family of cell wall proteins from S. cerevisiae has been exploited as a fusion protein for display because of its alternate linkage capabilities. In addition, a yeast surface display strategy based on cell wall protein Spi1 as an anchor may be employed according to the methods of the present disclosure. A non-limiting example of a yeast display platform that may be employed according to the methods of the present disclosure include the use of a glycosylphosphatidylinositol anchor sequence which covalently tethers the protein to the yeast cell wall, as described in McMahon et al. (2018) Nat Struct Mol Biol. 25(3):289-296. Further non-limiting examples of strategies for anchoring polypeptides to the yeast cell wall which may be employed according to the methods of the present disclosure include those described in Andreu & Olmo (2018) Appl Microbiol Biotechnol. 102(6):2543-2561, the disclosure of which is incorporated herein by reference in its entirety for all purposes. [[Jack: I don't have access to this paper, and my understanding is it describes Adimab's anchoring approach. Please provide a brief description of that approach here, or send a pdf of the Andreu & Olmo and I can supplement this section.]] Detailed guidance for performing yeast display which may be incorporated into the methods of the present disclosure for identifying a polypeptide that specifically binds domain 2 of a human LAG-3 polypeptide can be found, e.g., in Chao et al. (2006) Nature Protocols 1 (2): 755-768.

According to some embodiments, the polypeptides of the polypeptide library are displayed on one or more solid supports. In certain embodiments, the one or more solid supports comprise one or more wells. The one or more wells may be one or more wells of a plate, a microfluidic device, or the like. According to some embodiments, the one or more solid supports comprise particulate solid supports. In certain embodiments, the particulate solid supports are beads.

In certain embodiments, the polypeptides of the polypeptide library are complexed with ribosome-mRNA (RM) complexes during the contacting, and wherein the mRNAs encode the polypeptides of the polypeptide library. For example, a ribosome display methodology for identifying a polypeptide that specifically binds domain 2 of a human LAG-3 polypeptide may be employed as described above.

According to some embodiments, the polypeptides of the polypeptide library are complexed with DNAs during the contacting, and wherein the DNAs encode the polypeptides of the polypeptide library. For example, a DNA display methodology for identifying a polypeptide that specifically binds domain 2 of a human LAG-3 polypeptide may be employed, as described above.

In certain embodiments, the polypeptides of the polypeptide library are soluble during the contacting step. By “soluble” in this context means the polypeptides are untethered during the contacting step, e.g., not displayed on a surface (e.g., bead), cell, phage or the like during the contacting step.

According to some embodiments, each of the plurality of human LAG-3 polypeptides consist of the extracellular domain of a human LAG-3 polypeptide or a fragment thereof comprising domain 2. In certain embodiments, each of the plurality of human LAG-3 polypeptides consist of domains 1 and 2 of a human LAG-3 polypeptide or a fragment thereof comprising domain 2.

In certain embodiments, the polypeptide library is an antibody library. According to some embodiments, antibody library is a IgG library, a Fab library, an scFv library, or single variable domain located on a heavy chain (VHH) library. In certain embodiments, the antibody library is a human chimeric or fully human antibody library.

According to some embodiments, the polypeptide library is a knottin peptide library, a fibronectin type-III (FNIII) domain library (see, e.g., Olson & Roberts (2007) Protein Sci. 16(3):476-484), or a designed ankyrin repeat protein (DARPin) library (see, e.g., Hartmann et al. (2018) Mol Ther Methods Clin Dev 10:128-143). Non-limiting examples of knottin peptide libraries that may be employed according to the methods of the present disclosure include EETI-II peptide, AgRP peptide, ω-conotoxin peptide, Kalata B1 peptide, MCoTI-II peptide, agatoxin peptide, or chlorotoxin peptide libraries. The three-dimensional structure of a knottin peptide is minimally defined by a particular arrangement of three disulfide bonds. This characteristic topology forms a molecular knot in which one disulfide bond passes through a macrocycle formed by the other two intra-chain disulfide bridges. Although their secondary structure content is generally low, knottins share a small triple-stranded antiparallel β-sheet, which is stabilized by the disulfide bond framework. Folding and functional activity of knottins are often mediated by loop regions that are diverse in both length and amino acid composition. While three disulfide bonds are the minimum number that defines the fold of this family of peptides, knottins can also contain additional cysteine residues, yielding molecules with four or more disulfide bonds and additional constrained loops in their structure. The term “cystine” refers to a Cys residue in which the sulfur group is linked to another amino acid though a disulfide linkage; the term “cysteine” refers to the —SH (“half cystine”) form of the residue. Binding loop portions may be adjacent to cystines, such that there are no other intervening cystines in the primary sequence in the binding loop. The knottin peptide library may be based on a knottin peptide described in the online KNOTTIN database, which includes detailed amino acid sequence, structure, classification and function information for thousands of polypeptides identified as contain cystine-knot motifs. In some embodiments, the knottin peptide library is an Ecballium elaterium trypsin inhibitor II (EETI-II) peptide, agouti-related protein (AgRP) peptide, ω-conotoxin peptide, Kalata B1 peptide, MCoTI-II peptide, agatoxin peptide, or chlorotoxin peptide library. In some embodiments, the knottin peptide library is an EETI-II peptide library. In some embodiments, the knottin peptide library is an agouti-related protein (AgRP) peptide library.

In certain embodiments, the screening comprises identifying, among the identified polypeptides that bind to the human LAG-3 polypeptides, polypeptides that bind to a human LAG-3 polypeptide consisting of domain 2 or a fragment thereof. For example, the screening may comprise a binding assay for determining which polypeptides bind to a human LAG-3 fragment consisting of domain 2 or a fragment thereof, e.g., a human LAG-3 fragment consisting of amino acids 175 to 230 of the human LAG-3 polypeptide set forth in SEQ ID NO:1 or a fragment thereof comprising amino acids I186, E215 and H220, wherein numbering is according to SEQ ID NO:1. Alternatively, or additionally, the screening may comprise a negative selection procedure to eliminate polypeptides that bind to a domain of the human LAG-3 polypeptide other than domain 2. Such a negative selection procedure may comprise, e.g., a binding assay for determining which polypeptides bind to a human LAG-3 fragment which does not comprise domain 2, and eliminating those polypeptides (e.g., eliminating phage, yeast, or the like encoding such polypeptides). According to some embodiments, the screening comprises a negative selection procedure to eliminate polypeptides that bind to a region of the human LAG-3 polypeptide other than a region within amino acids 175 to 230 of the human LAG-3 polypeptide set forth in SEQ ID NO: 1. For example, in certain embodiments, a negative selection procedure comprises a binding assay for determining which polypeptides that retain binding to a human LAG-3 fragment that comprises domain 2 or a portion thereof comprising an amino acid substitution (e.g., alanine substitution) at one or more of I186, E215 and H220, which amino acids have been determined by the inventors to play a role in human LAG-3 dimerization. Polypeptides that bind to such an amino acid substituted domain 2 of human LAG-3 may then be identified as polypeptides which are not likely to disrupt dimerization of human LAG-3, and may be eliminated on that basis. Screening assays for positively or negatively selecting for antibodies based on antibody binding properties are known and include enzyme-linked immunosorbent assay (ELISA), flow cytometry (e.g., fluorescence-activated cell sorting (FACS)), the gel encapsulated microdroplet (GEM) assay (Crystal Bioscience/Ligand Pharmaceuticals), the xPloration™ platform (Ligand Pharmaceuticals), the high-throughput single-cell screening platform of AbCellera Biologics Inc., the Beacon® high-throughput single-cell screening platform of Berkeley Lights, Inc., and the like.

In certain embodiments, the polypeptide library is a chimeric or fully human polypeptide library.

According to some embodiments, the in vitro methods further comprise screening the polypeptides identified as specifically binding domain 2 of the human LAG-3 polypeptide for the ability to disrupt human LAG-3 dimerization. The screening may comprise, e.g., combining human LAG-3 monomers and/or dimers with the polypeptides identified as specifically binding domain 2 of the human LAG-3 polypeptide (or when the polypeptides are antibodies, antibodies comprising the six CDRs of the polypeptides) and assessing for the presence or absence of human LAG-3 dimers, e.g., by sodium dodecyl sulphate-polyacrylamide gel electrophoresis (SDS-PAGE), size exclusion chromatography, analytical ultracentrifugation, electron microscopy (see, e.g., Example 2 and FIG. 3 herein), or the like. The methods may further comprise identifying a polypeptide that disrupts LAG-3 dimerization as a LAG-3 dimerization disrupting agent.

### In Silico Screening Methods

Aspects of the present disclosure further include methods of identifying an agent as a candidate human LAG-3 dimerization disrupting agent. Such methods comprise conducting an in silico screen to identify an agent that interacts with the domain 2 dimerization interface of human LAG-3, wherein an agent that interacts with the domain 2 dimerization interface of human LAG-3 is a candidate human LAG-3 dimerization disrupting agent. Also provided by the present disclosure are methods of identifying an agent as a candidate human LAG-3 dimerization disrupting agent, such methods comprising conducting an in silico screen to identify an agent that interacts with a domain of human LAG-3 other than domain 2 (e.g., domain 1 or domain 3) and is expected to disrupt dimerization of human LAG-3 via an allosteric effect on the domain 2 dimerization interface, wherein an agent that interacts with a domain of human LAG-3 other than domain 2 and is expected to disrupt dimerization of human LAG-3 via an allosteric effect on the domain 2 dimerization interface is a candidate human LAG-3 dimerization disrupting agent. Small molecule and polypeptide libraries, in silico screening approaches, etc. that find use in practicing such methods are described hereinbelow.

As used herein, an “in silico screen” means a filtering of large databases or libraries of possible agents through the use of computational approaches based on discrimination functions that permit the selection of agents to be tested for biological activity. An exemplary in silico screen according to the present disclosure uses an in silico small molecule or polypeptide library. Screening may be performed using FRED2.0 and/or the like. Docking poses may be scored by PLP. Other approaches to in silico screens are known in the art. See, e.g., Plewcznyski et al., Chem. Biol. Drug. Res., 69(4):269-79 (2007), Lu et al., J. Med. Chem., 49(17):5154-61 (2006), Nicolazzo et al., J. Pharm. Pharmacol., 58(3):281-93 (2006), and Langer and Wolber, Pure Appl. Chem., 76(5):991-996 (2004). As used herein, to “interact”, means to have a relationship, for example, by forming hydrogen bonds, by hydrophobic stacking, by having cation-pi interactions, and/or the like.

Non-limiting examples of approaches that may be implemented for in silico screening of small molecule libraries for candidate small molecule human LAG-3 dimerization disrupting agents according to the methods of the present disclosure include those described in Bender et al. (2021) Nat Protoc 16:4799-4832; Tran-Nguyen et al. (2021) J. Chem. Inf. Model. 61(6):2788-2797; and Cleves & Jain (2020) Chem. Inf. Model. 60(9):4296-4310; the disclosures of which are incorporated herein by reference in their entireties for all purposes.

Non-limiting examples of approaches that may be implemented for in silico screening of polypeptide libraries for candidate polypeptide human LAG-3 dimerization disrupting agents according to the methods of the present disclosure include those described in Cao et al. (2020) Science 370(6515):426-431; Cao et al. (2021) Robust de novo design of protein binding proteins from target structural information alone (doi.org/10.1101/2021.09.04.459002); Eguchi et al. (2020) IG-VAE: Generative Modeling of Immunoglobulin Proteins by Direct 3D Coordinate Generation (doi.org/10.1101/2020.08.07.242347); and Strokach & Kim (2021) Deep Generative Modeling for Protein Design (arXiv: 2109.13754); the disclosures of which are incorporated herein by reference in their entireties for all purposes.

In certain embodiments, the conducting comprises conducting an in silico screen to identify an agent that interacts with an amino acid of the domain 2 dimerization interface of human LAG-3 selected from the group consisting of: A181, S182, W184, I186, N188, R196, E215, H220, A222, E223. F225, F227, P229, Q230, and any combination thereof, wherein numbering is according to SEQ ID NO:1. According to some embodiments, the conducting comprises conducting an in silico screen to identify an agent that interacts with an amino acid of the domain 2 dimerization interface of human LAG-3 selected from the group consisting of: I186, E215, H220, E223, and any combination thereof.

According to some embodiments, the conducting comprises conducting an in silico screen to identify an agent that interacts with amino acid I186 of the domain 2 dimerization interface of human LAG-3. According to some such embodiments, the conducting comprises conducting an in silico screen to identify an agent that interferes with interaction between amino acid I186 of the domain 2 dimerization interface of human LAG-3 and one or each of amino acids A222 and F225 in the opposite monomer of a human LAG-3 dimer, wherein numbering is according to SEQ ID NO: 1.

In certain embodiments, the conducting comprises conducting an in silico screen to identify an agent that interacts with amino acid E215 of the domain 2 dimerization interface of human LAG-3. According to some such embodiments, the conducting comprises conducting an in silico screen to identify an agent that interferes with interaction between amino acid E215 of the domain 2 dimerization interface of human LAG-3 and one or more amino acids in the opposite monomer of a human LAG-3 dimer.

According to some embodiments, the conducting comprises conducting an in silico screen to identify an agent that interacts with amino acid H220 of the domain 2 dimerization interface of human LAG-3. According to some such embodiments, the conducting comprises conducting an in silico screen to identify an agent that interferes with interaction between amino acid H220 of the domain 2 dimerization interface of human LAG-3 and one or each of amino acids W184 and I186 in the opposite monomer of a human LAG-3 dimer, wherein numbering is according to SEQ ID NO: 1.

In certain embodiments, the conducting comprises conducting an in silico screen to identify an agent that interacts with amino acid E223 of the domain 2 dimerization interface of human LAG-3. According to some such embodiments, the conducting comprises conducting an in silico screen to identify an agent that interferes with interaction between amino acid E223 of the domain 2 dimerization interface of human LAG-3 and one, two or each of amino acids N188 glycan, E223 and F225 in the opposite monomer of a human LAG-3 dimer, wherein numbering is according to SEQ ID NO:1.

According to some embodiments, the agent is a small molecule. By “small molecule” compound is meant a compound having a molecular weight of 1000 atomic mass units (amu) or less. In some embodiments, the small molecule is 900 amu or less, 750 amu or less, 500 amu or less, 400 amu or less, 300 amu or less, or 200 amu or less. In certain aspects, the small molecule is not made of repeating molecular units such as are present in a polymer.

In certain embodiments, the agent is a polypeptide. The terms “peptide,” “polypeptide,” and “protein” are used interchangeably herein. In the present invention, these terms mean a linked sequence of amino acids, which may be natural, synthetic, or a modification or combination of natural and synthetic. According to some embodiments, the agent is an antibody.

Any of the in silico screening methods of the present disclosure may further comprise screening the candidate human LAG-3 dimerization disrupting agent for the ability to disrupt human LAG-3 dimerization. Such methods may further comprise identifying a candidate human LAG-3 dimerization disrupting agent that disrupts LAG-3 dimerization as a LAG-3 dimerization disrupting agent. The screening may comprise, e.g., combining human LAG-3 monomers and/or dimers with the candidate agent and assessing for the presence or absence of human LAG-3 dimers, e.g., by sodium dodecyl sulphate-polyacrylamide gel electrophoresis (SDS-PAGE), size exclusion chromatography, analytical ultracentrifugation, electron microscopy (see, e.g., Example 2 and FIG. 3 herein), or the like.

### Polypeptides, Nucleic Acids and Cells

Aspects of the present disclosure further include polypeptides (e.g., monoclonal antibodies, etc.) that specifically bind domain 2 of a human LAG-3 polypeptide generated according to the in vivo methods of the present disclosure or identified according to the in vitro methods of the present disclosure. In some embodiments, such polypeptides are those identified as specifically binding a human LAG3 polypeptide within amino acids 175 to 230 of the human LAG-3 polypeptide set forth in SEQ ID NO:1.

Also provided are monoclonal antibodies comprising the six CDRs of a monoclonal antibody generated according to the in vivo methods of the present disclosure or identified according to the in vitro methods of the present disclosure. In certain embodiments, such a monoclonal antibody is an IgG antibody. e.g., a human IgG1 or IgG4 antibody. According to some embodiments, the monoclonal antibody is a Fab, a F(ab′)₂, or a F(ab′). In certain embodiments, the monoclonal antibody is a single chain antibody, e.g., an scFv.

According to some embodiments, the monoclonal antibody is a bispecific antibody or a multi-specific antibody comprising a first antigen-binding domain that specifically binds domain 2 of a human LAG-3 polypeptide and a second antigen-binding domain. In certain embodiments, the second antigen-binding domain specifically binds domain 2 of a human LAG-3 polypeptide, and the epitope of domain 2 bound by the second antigen-binding domain is different from the epitope of domain 2 bound by the first antigen-binding domain. In other embodiments, the second antigen-binding domain specifically binds a domain of the human LAG-3 polypeptide other than domain 2. In still other embodiments, the second antigen-binding domain specifically binds an antigen other than the human LAG-3 polypeptide.

The “knob-in-hole” strategy (see, e.g., PCT Intl. Publ. No. WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. U82010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351 Y_F405A_Y407V T394W, T3661_K392 M_T394W/F405A_Y407V, T366L_K392 M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in U.S. Pat. Pubi. No. US2012/0149876 or U.S. Pat. Publ. No. US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8 (18): 3689-3696.

In certain embodiments, a monoclonal antibody of the present disclosure is a pH-selective antibody, a protease-activated antibody, or a pH-selective and protease-activated antibody.

### Methods of Producing Polypeptides

Using the information provided herein, a polypeptide that specifically binds domain 2 of a human LAG-3 polypeptide of the present disclosure may be prepared using techniques known to those of skill in the art. For example, a nucleic acid sequence encoding the amino acid sequence of an antibody of the present disclosure can be used to express the antibodies. The nucleic acid sequence(s) can be optimized to reflect particular codon “preferences” for various expression systems according to methods known to those of skill in the art.

The nucleic acids may be synthesized according to a number of standard methods known to those of skill in the art. Oligonucleotide synthesis, is preferably carried out on commercially available solid phase oligonucleotide synthesis machines or manually synthesized using, for example, a solid phase phosphoramidite triester method.

Once a nucleic acid encoding a subject antibody is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies of the present disclosure can be achieved by operably linking a nucleic acid encoding the antibody to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp, and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject antibody can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody can be further purified by dialysis and other methods normally employed in protein purification methods. In one embodiment, the antibody may be isolated using metal chelate chromatography methods. Antibodies of the present disclosure may contain modifications to facilitate isolation, as discussed above.

The subject antibodies may be prepared in substantially pure or isolated form (e.g., free from other polypeptides). The protein can be present in a composition that is enriched for the polypeptide relative to other components that may be present (e.g., other polypeptides or other host cell components). Purified antibodies may be provided such that the antibody is present in a composition that is substantially free of other expressed proteins, e.g., less than 90%, usually less than 60% and more usually less than 50% of the composition is made up of other expressed proteins.

The antibodies produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the antibodies may be operably linked to a secretion signal sequence such as pelB so that the antibodies are secreted into the periplasm in correctly-folded form.

The present disclosure also provides cells that produce the antibodies of the present disclosure, where suitable cells include eukaryotic cells, e.g., mammalian cells. The cells can be a hybrid cell or “hybridoma” that is capable of reproducing antibodies in vitro (e.g. monoclonal antibodies, such as IgG). For example, the present disclosure provides a recombinant host cell (also referred to herein as a “genetically modified host cell”) that is genetically modified with one or more nucleic acids comprising a nucleotide sequence encoding a heavy and/or light chain of an antibody of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia) insect or mammalian (e.g., CHO cell) expression system, for example. One example of a suitable technique uses a bacteriophage lambda vector system having a leader sequence that causes the expressed antibody (e.g., Fab or scFv) to migrate to the periplasmic space (between the bacterial cell membrane and the cell wall) or to be secreted. One can rapidly generate great numbers of functional fragments (e.g., Fab or scFv) for those which bind the antigen. In certain embodiments, a cell-free protein expression system is employed.

### Nucleic Acids, Expression Vectors and Cells

In view of the section above regarding methods of producing the antibodies of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an antibody of the present disclosure.

In certain embodiments, provided is a nucleic acid encoding a variable heavy chain (V_{H}) polypeptide, a variable light chain (V_{L}) polypeptide, or both, of an antibody of the present disclosure, wherein the antibody is a single chain antibody (e.g., an scFv), and the nucleic acid encodes the single chain antibody.

Any of the nucleic acids of the present disclosure may be operably linked to a heterologous expression control sequence, e.g., a heterologous promoter.

Also provided are expression vectors comprising any of the nucleic acids of the present disclosure.

Cells that comprise any of the nucleic acids and/or expression vectors of the present disclosure are also provided. Such cells include prokaryotic cells and eukaryotic cells. Eukaryotic cells of interest include yeast cells, mammalian cells (e.g., murine cells, human cells, or the like), etc. According to some embodiments, a cell of the present disclosure includes a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such a cell comprises a first expression vector comprising the first nucleic acid, and a second expression vector comprising the second nucleic acid.

Any of the cells of the present disclosure may comprise the nucleic acid(s) operably linked to a heterologous expression control sequence, e.g., a heterologous promoter.

Also provided are methods of making an antibody of the present disclosure, the methods including culturing a cell of the present disclosure under conditions suitable for the cell to express the antibody, wherein the antibody is produced. The conditions suitable for the cell to express the antibody may vary. Non-limiting examples of such conditions include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C. such as 37° C.) and pH (e.g., pH 7.0-7.7, such as pH 7.4) in an environment having a suitable percentage of CO₂, e.g., from 3% to 10%, such as 5%.

### Compositions

The present disclosure also provides compositions. According to some embodiments, a composition of the present disclosure comprises a polypeptide of the present disclosure. For example, the polypeptide may be any of the monoclonal antibodies described hereinabove, which is incorporated but not reiterated herein for purposes of brevity. In certain embodiments, a composition of the present disclosure comprises an agent of the present disclosure (e.g., a LAG-3 dimerization disrupting agent) identified according to the methods of the present disclosure.

In certain embodiments, a composition of the present disclosure includes the polypeptide or agent present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure include pharmaceutical compositions. In some embodiments, a pharmaceutical composition of the present disclosure includes polypeptide or agent of the present disclosure and a pharmaceutically acceptable carrier.

Formulations of the monoclonal antibodies and agents (collectively referred to as “agent(s)” below) for administration to the individual (e.g., suitable for human administration) are generally sterile and may further be free of detectable pyrogens or other contaminants contraindicated for administration to a patient according to a selected route of administration.

In pharmaceutical dosage forms, the agent(s) can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the agent(s) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent(s) can be formulated for parenteral (e.g., intravenous, intra-arterial, intraosseous, intramuscular, intracerebral, intracerebroventricular, intrathecal, subcutaneous, etc.) administration. In certain aspects, the agent(s) are formulated for injection by dissolving, suspending or emulsifying the agent(s) in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent(s) may be prepared by mixing the agent(s) having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

An aqueous formulation of the agent(s) may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylenesorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the agent(s) against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the agent(s), and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods of Use

Aspects of the present disclosure further include methods comprising administering an effective amount of a pharmaceutical composition of the present disclosure to an individual in need thereof.

In certain embodiments, provided are methods of administering an anti-LAG3 immunotherapy to an individual in need thereof, the method comprising administering an effective amount of a pharmaceutical composition of the present disclosure to an individual in need thereof. According to some embodiments, the anti-LAG3 immunotherapy is administered as a monotherapy. In certain embodiments, the anti-LAG3 immunotherapy does not comprise administering an agent that targets an immune checkpoint molecule other than LAG-3 to the individual. According to some embodiments, the anti-LAG3 immunotherapy does not comprise administering an anti-PD1 agent to the individual. In certain embodiments, the anti-LAG3 immunotherapy comprises administering an anti-PD1 agent to the individual.

According to some embodiments, the pharmaceutical composition comprises an antibody that specifically binds domain 1, 2 or 3 of human LAG-3 and disrupts human LAG-3 dimerization. In certain embodiments, the antibody comprises, or competes for binding to domain 2 of human LAG-3 with an antibody comprising, the six complementarity determining regions (CDRs) of antibody C9B7W. According to some embodiments, the antibody comprises, or competes for binding to domain 1 of human LAG-3 with an antibody comprising, the six CDRs of antibody M8-4-6. In some instances, the antibody comprises, or competes for binding to domain 3 of human LAG-3 with an antibody comprising, the six CDRs of antibody 410C9.

In certain embodiments, the individual has a cell proliferative disorder, and the anti-LAG3 immunotherapy is effective in treating the cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the individual has cancer, and the anti-LAG3 immunotherapy is effective in treating the cancer. In some embodiments, the subject has a cancer suspected of evading the immune system (e.g., effector T cells), e.g., by co-opting one or more immune checkpoint pathways. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance: benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma: brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polyploid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or ureter); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas. In some embodiments, the cancer is a hematologic malignancy. Non-limiting examples of hematologic malignancies include leukemias, lymphomas, or multiple myeloma.

The pharmaceutical composition may be administered to any of a variety of subjects. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain embodiments, the individual is an animal model (e.g., a mouse model, a primate model, or the like).

The pharmaceutical composition is administered in a therapeutically effective amount. By “therapeutically effective amount” is meant a dosage sufficient to produce a desired result, e.g., an amount sufficient to effect beneficial or desired therapeutic (including prophylactic) results, such as the prevention or a reduction in a symptom of a cell proliferative disorder (e.g., cancer), as compared to a control. In some embodiments, the therapeutically effective amount is sufficient to slow the progression of, or reduce, one or more symptoms of a cell proliferative disorder (e.g., cancer). According to some embodiments, the therapeutically effective amount slows the progression of, or reduces, one or more of such symptoms by 10% or more, 20% or more, 30% or more, 40% or more, 50% or more, 60% or more, 70% or more, 80% or more, 90% or more, or 100% or more, as compared to the one or more symptoms in the absence of the administration of the pharmaceutical composition. An effective amount can be administered in one or more administrations.

The pharmaceutical composition may be administered via a route of administration independently selected from oral, parenteral (e.g., by intravenous, intra-arterial, subcutaneous, intramuscular, or epidural injection), topical, or nasal administration. According to certain embodiments, the pharmaceutical composition is administered parenterally.

### Kits

Aspects of the present disclosure further include kits. The kits find use, e.g., in practicing the methods of the present disclosure. In some embodiments, a subject kit includes a composition (e.g., a pharmaceutical composition) that includes any of the monoclonal antibodies, polypeptides and/or agents of the present disclosure. In some embodiments, provided are kits that include any of the pharmaceutical compositions described herein, including any of the pharmaceutical compositions described above in the section relating to the compositions of the present disclosure. Kits of the present disclosure may include instructions for administering the pharmaceutical composition to an individual in need thereof, including but not limited to, an individual having a cell proliferative disorder, e.g., cancer.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes any of the monoclonal antibodies and/or agents of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular polypeptide and/or agent employed, the effect to be achieved, and the pharmacodynamics associated with the polypeptide and/or agent in the individual. In yet other embodiments, the kits may include a single multi dosage amount of the composition.

As will be appreciated, the kits of the present disclosure may include any of the agents and features described above in the sections relating to the subject antibodies, agents, methods and compositions, which are not reiterated in detail herein for purposes of brevity.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:
1. A method of generating cells that produce monoclonal antibodies that specifically bind domain 2 of a human lymphocyte activation gene-3 (LAG-3) polypeptide, the method comprising:
   immunizing a non-human animal with an immunogen comprising domain 2 of a human LAG-3 polypeptide;
   isolating monoclonal antibody-producing cells from the non-human animal, wherein the monoclonal antibody-producing cells produce monoclonal antibodies that specifically bind the immunogen; and
   screening the monoclonal antibody-producing cells for cells that produce monoclonal antibodies that specifically bind domain 2 of the human LAG-3 polypeptide.
2. The method according to embodiment 1, wherein the immunogen consists of the extracellular domain of a human LAG-3 polypeptide or a fragment thereof comprising domain 2.
3. The method according to embodiment 1 or embodiment 2, wherein the immunogen consists of domains 1 and 2 of a human LAG-3 polypeptide or a fragment thereof comprising domain 2.
4. The method according to any one of embodiments 1 to 3, wherein the immunizing comprises administering the immunogen to the non-human animal.
5. The method according to any one of embodiments 1 to 3, wherein the immunizing comprises administering a nucleic acid encoding the immunogen to the non-human animal, wherein the nucleic acid is configured to express the immunogen upon administration to the non-human animal.
6. The method according to any one of embodiments 1 to 3, wherein the immunizing comprises administering cells that express the immunogen to the non-human animal, wherein the cells secrete the immunogen and/or display the immunogen on their surface upon administration to the non-human animal.
7. The method according to any one of embodiments 1 to 6, wherein the screening comprises identifying, among the monoclonal antibody-producing cells, cells that produce monoclonal antibodies that bind to a human LAG-3 polypeptide consisting of domain 2 or a fragment thereof.
8. The method according to embodiment 7, wherein the screening comprises identifying, among the monoclonal antibody-producing cells, cells that produce monoclonal antibodies that bind to a human LAG-3 polypeptide consisting of amino acids 175 to 230 of the human LAG-3 polypeptide set forth in SEQ ID NO:1 or a fragment thereof comprising amino acids I186, E215 and H220, wherein numbering is according to SEQ ID NO:1.
9. The method according to any one of embodiments 1 to 8, wherein the screening comprises a negative selection procedure to eliminate cells that produce monoclonal antibodies that bind to a domain of the human LAG-3 polypeptide other than domain 2.
10. The method according to any one of embodiments 1 to 8, wherein the screening comprises a negative selection procedure to eliminate cells that produce monoclonal antibodies that bind to a region of the human LAG-3 polypeptide other than a region within amino acids 175 to 230 of the human LAG-3 polypeptide set forth in SEQ ID NO:1.
11. The method according to any one of embodiments 1 to 10, wherein the screening comprises performing single cell sequencing on the monoclonal antibody-producing cells, determining the sequences of the antibodies produced by the monoclonal antibody-producing cells, and identifying cells as candidate cells that produce monoclonal antibodies that specifically bind domain 2 of the human LAG-3 polypeptide based on the sequences.
12. The method according to any one of embodiments 1 to 11, wherein the non-human animal is selected from the group consisting of: a mouse, a rat, a chicken, a cow, a goat, a llama, an alpaca, a shark, and a rabbit.
13. The method according to any one of embodiments 1 to 12, wherein the non-human animal comprises a replacement of one or more endogenous non-human animal immunoglobulin (Ig) genes with human Ig genes.
14. The method according to embodiment 13, wherein the monoclonal antibody-producing cells produce human/non-human animal chimeric monoclonal antibodies or fully human monoclonal antibodies that specifically bind the immunogen.
15. The method according to any one of embodiments 1 to 13, wherein the method further comprises screening monoclonal antibodies identified as specifically binding domain 2 of the human LAG-3 polypeptide for the ability to disrupt human LAG-3 dimerization.
16. The method according to embodiment 15, further comprising identifying a monoclonal antibody that disrupts LAG-3 dimerization as a LAG-3 dimerization disrupting agent.
17. The method according to any one of embodiments 1 to 16, further comprising immortalizing cells that produce monoclonal antibodies that specifically bind domain 2 of the human LAG-3 polypeptide.
18. A cell that produces a monoclonal antibody that specifically binds domain 2 of a human LAG-3 polypeptide obtained according to the method of any one of embodiments 1 to 17.
9. The cell of embodiment 18, wherein the monoclonal antibody specifically binds a human LAG-3 polypeptide within amino acids 175 to 230 of the human LAG-3 polypeptide set forth in SEQ ID NO:1.
20. A monoclonal antibody comprising the six complementarity determining regions (CDRs) of a monoclonal antibody produced by the cell of embodiment 18 or embodiment 19.
21. A method of identifying a polypeptide that specifically binds domain 2 of a human LAG-3 polypeptide, the method comprising:
   contacting a plurality of human LAG-3 polypeptides with a polypeptide library, wherein each of the plurality of human LAG-3 polypeptides comprise domain 2 of human LAG-3;
   identifying polypeptides of the polypeptide library that specifically bind to human LAG-3 polypeptides of the plurality of human LAG-3 polypeptides; and
   screening the polypeptides that bind to the human LAG-3 polypeptides for a monoclonal antibody that specifically binds domain 2 of human LAG-3.
22. The method according to embodiment 21, wherein the human LAG-3 polypeptides of the plurality are displayed on the surface of cells.
23. The method according to embodiment 22, wherein the cells are mammalian cells.
24. The method according to embodiment 22, wherein the cells are yeast cells.
25. The method according to embodiment 21, wherein the human LAG-3 polypeptides of the plurality are displayed on one or more solid supports.
26. The method according to embodiment 25, wherein the human LAG-3 polypeptides of the plurality are displayed on the surface of one or more wells.
27. The method according to embodiment 25, wherein the one or more solid supports comprise particulate solid supports.
28. The method according to embodiment 27, wherein the particulate solid supports comprise beads.
29. The method according to embodiment 21, wherein the human LAG-3 polypeptides of the plurality are complexed with ribosome-mRNA (RM) complexes during the contacting, and wherein the mRNAs encode the human LAG-3 polypeptides.
30. The method according to embodiment 21, wherein the human LAG-3 polypeptides of the plurality are complexed with DNAs during the contacting, and wherein the DNAs encode the human LAG-3 polypeptides.
31. The method according to embodiment 21, wherein the human LAG-3 polypeptides of the plurality are soluble during the contacting step.
32. The method according to any one of embodiments 21 to 31, wherein the polypeptides of the polypeptide library are displayed on the surface of phage during the contacting.
33. The method according to any one of embodiments 21 to 31, wherein the polypeptides of the polypeptide library are displayed on the surface of cells during the contacting.
34. The method according to embodiment 33, wherein the cells are mammalian cells.
35. The method according to embodiment 33, wherein the cells are yeast cells.
36. The method according to any one of embodiments 21 to 31, wherein the polypeptides of the polypeptide library are displayed on one or more solid supports.
37. The method according to embodiment 36, wherein the polypeptides of the polypeptide library are displayed on the surface of one or more wells.
38. The method according to embodiment 36, wherein the one or more solid supports comprise particulate solid supports.
39. The method according to embodiment 38, wherein the particulate solid supports comprise beads.
40. The method according to embodiment 38 or embodiment 39, wherein the particulate solid supports are magnetically responsive.
41. The method according to any one of embodiments 21 to 31, wherein the polypeptides of the polypeptide library are complexed with ribosome-mRNA (RM) complexes during the contacting, and wherein the mRNAs encode the polypeptides of the polypeptide library.
42. The method according to any one of embodiments 21 to 31, wherein the polypeptides of the polypeptide library are complexed with DNAs during the contacting, and wherein the DNAs encode the polypeptides of the polypeptide library.
43. The method according to any one of embodiments 21 to 31, wherein the polypeptides of the polypeptide library are soluble during the contacting step.
44. The method according to any one of embodiments 21 to 43, wherein each of the plurality of human LAG-3 polypeptides consist of the extracellular domain of a human LAG-3 polypeptide or a fragment thereof comprising domain 2.
45. The method according to any one of embodiments 21 to 43, wherein each of the plurality of human LAG-3 polypeptides consist of domains 1 and 2 of a human LAG-3 polypeptide or a fragment thereof comprising domain 2.
46. The method according to any one of embodiments 21 to 45, wherein the screening comprises identifying, among the identified polypeptides that bind to the human LAG-3 polypeptides, polypeptides that bind to a human LAG-3 polypeptide consisting of amino acids 175 to 230 of the human LAG-3 polypeptide set forth in SEQ ID NO:1 or a fragment thereof comprising amino acids I186, E215 and H220, wherein numbering is according to SEQ ID NO: 1.
47. The method according to any one of embodiments 21 to 46, wherein the screening comprises a negative selection procedure to eliminate polypeptides that bind to a domain of the human LAG-3 polypeptide other than domain 2.
48. The method according to any one of embodiments 21 to 46, wherein the screening comprises a negative selection procedure to eliminate polypeptides that bind to a region of the human LAG-3 polypeptide other than a region within amino acids 175 to 230 of the human LAG-3 polypeptide set forth in SEQ ID NO:1.
49. The method according to any one of embodiments 21 to 48, wherein the polypeptide library is an antibody library.
50. The method according to embodiment 49, wherein the antibody library is a IgG library, a Fab library, an scFv library, or single variable domain located on a heavy chain (VHH) library.
51. The method according to embodiment 49 or embodiment 50, wherein the antibody library is a human chimeric or fully human antibody library.
52. The method according to any one of embodiments 21 to 48, wherein the polypeptide library is a knottin library, a fibronectin type-III (FNIII) domain library, or a designed ankyrin repeat protein (DARPin) library.
53. The method according to any one of embodiments 21 to 51, wherein the method further comprises screening polypeptides identified as specifically binding domain 2 of the human LAG-3 polypeptide for the ability to disrupt human LAG-3 dimerization.
54. The method according to embodiment 53, further comprising identifying a polypeptide that disrupts LAG-3 dimerization as a LAG-3 dimerization disrupting agent.
55. A polypeptide that specifically binds domain 2 of a human LAG-3 polypeptide identified according to the method of any one of embodiments 21 to 54.
6. The polypeptide of embodiment 55, wherein the polypeptide specifically binds a human LAG-3 polypeptide within amino acids 175 to 230 of the human LAG-3 polypeptide set forth in SEQ ID NO:1.
57. The polypeptide of embodiment 55 or embodiment 56, wherein the polypeptide is an antibody.
58. A monoclonal antibody comprising the six CDRs of the antibody of embodiment 57.
59. The monoclonal antibody of embodiment 20 or embodiment 58, wherein the monoclonal antibody is an IgG.
60. The monoclonal antibody of embodiment 20 or embodiment 58, wherein the monoclonal antibody is selected from the group consisting of: a Fab, a F(ab′)₂, and a F(ab′).
61. The monoclonal antibody of embodiment 20 or embodiment 58, wherein the monoclonal antibody is a single chain antibody, optionally wherein the single chain antibody is an scFv.
62. The monoclonal antibody of any one of embodiments 20 or 58 to 61, wherein the monoclonal antibody is a bispecific antibody or a multi-specific antibody comprising a first antigen-binding domain that specifically binds domain 2 of a human LAG-3 polypeptide and a second antigen-binding domain.
63. The monoclonal antibody of embodiment 62, wherein the second antigen-binding domain specifically binds domain 2 of a human LAG-3 polypeptide, and wherein the epitope of domain 2 bound by the second antigen-binding domain is different from the epitope of domain 2 bound by the first antigen-binding domain.
64. The monoclonal antibody of embodiment 62, wherein the second antigen-binding domain specifically binds a domain of the human LAG-3 polypeptide other than domain 2.
65. The monoclonal antibody of embodiment 62, wherein the second antigen-binding domain specifically binds an antigen other than the human LAG-3 polypeptide.
66. The monoclonal antibody of any one of embodiments 20 or 58 to 65, wherein the monoclonal antibody is a pH-selective antibody, a protease-activated antibody, or a pH-selective and protease-activated antibody.
67. A method of identifying an agent as a candidate human LAG-3 dimerization disrupting agent, the method comprising:
   conducting an in silico screen to identify an agent that interacts with the domain 2 dimerization interface of human LAG-3, wherein an agent that interacts with the domain 2 dimerization interface of human LAG-3 is a candidate human LAG-3 dimerization disrupting agent.
68 The method according to embodiment 67, wherein the conducting comprises conducting an in silico screen to identify an agent that interacts with an amino acid of the domain 2 dimerization interface of human LAG-3 selected from the group consisting of: A181, S182, W184, I186, N188, R196, E215, H220, A222, E223, F225, F227, P229, Q230, and any combination thereof, wherein numbering is according to SEQ ID NO:1.
69. The method according to embodiment 68, wherein the conducting comprises conducting an in silico screen to identify an agent that interacts with an amino acid of the domain 2 dimerization interface of human LAG-3 selected from the group consisting of: I186, E215, H220, E223, and any combination thereof.
70. The method according to embodiment 69, wherein the conducting comprises conducting an in silico screen to identify an agent that interacts with amino acid I186 of the domain 2 dimerization interface of human LAG-3.
71. The method according to embodiment 70, wherein the conducting comprises conducting an in silico screen to identify an agent that interferes with interaction between amino acid I186 of the domain 2 dimerization interface of human LAG-3 and one or each of amino acids A222 and F225 in the opposite monomer of a human LAG-3 dimer, wherein numbering is according to SEQ ID NO:1.
72. The method according to embodiment 69, wherein the conducting comprises conducting an in silico screen to identify an agent that interacts with amino acid E215 of the domain 2 dimerization interface of human LAG-3.
73. The method according to embodiment 74, wherein the conducting comprises conducting an in silico screen to identify an agent that interferes with interaction between amino acid E215 of the domain 2 dimerization interface of human LAG-3 and one or more amino acids in the opposite monomer of a human LAG-3 dimer.
74. The method according to embodiment 69, wherein the conducting comprises conducting an in silico screen to identify an agent that interacts with amino acid H220 of the domain 2 dimerization interface of human LAG-3.
75. The method according to embodiment 74, wherein the conducting comprises conducting an in silico screen to identify an agent that interferes with interaction between amino acid H220 of the domain 2 dimerization interface of human LAG-3 and one or each of amino acids W184 and I186 in the opposite monomer of a human LAG-3 dimer, wherein numbering is according to SEQ ID NO:1.
76. The method according to embodiment 69, wherein the conducting comprises conducting an in silico screen to identify an agent that interacts with amino acid E223 of the domain 2 dimerization interface of human LAG-3.
77. The method according to embodiment 76, wherein the conducting comprises conducting an in silico screen to identify an agent that interferes with interaction between amino acid E223 of the domain 2 dimerization interface of human LAG-3 and one, two or each of amino acids N188 glycan, E223 and F225 in the opposite monomer of a human LAG-3 dimer, wherein numbering is according to SEQ ID NO:1.
78. The method according to any one of embodiments 67 to 77, wherein the agent is a small molecule.
79. The method according to any one of embodiments 67 to 77, wherein the agent is a polypeptide.
80. The method according to any one of embodiments 67 to 77, wherein the agent is an antibody.
81. The method according to any one of embodiments 67 to 80, wherein the method further comprises screening the candidate human LAG-3 dimerization disrupting agent for the ability to disrupt human LAG-3 dimerization.
82. The method according to embodiment 81, further comprising identifying a candidate human LAG-3 dimerization disrupting agent that disrupts LAG-3 dimerization as a LAG-3 dimerization disrupting agent.
83. A pharmaceutical composition, comprising:
   the monoclonal antibody of embodiment 20 or 58 to 66; and
   a pharmaceutically acceptable carrier.
84. A pharmaceutical composition, comprising:
   the polypeptide according to any one of embodiments 55 to 66; and
   a pharmaceutically acceptable carrier.
85. A pharmaceutical composition, comprising:
   a LAG-3 dimerization disrupting agent identified according to the method of embodiment 82; and
   a pharmaceutically acceptable carrier.
86. A pharmaceutical composition, comprising:
   an antibody that comprises, or competes for binding to domain 2 of human LAG-3 with an antibody comprising, the six complementarity determining regions (CDRs) of antibody C9B7W; and
   a pharmaceutically acceptable carrier.
87. A pharmaceutical composition, comprising:
   an antibody that comprises, or competes for binding to domain 1 of human LAG-3 with an antibody comprising, the six CDRs of antibody M8-4-6; and
   a pharmaceutically acceptable carrier.
88. A pharmaceutical composition, comprising:
   an antibody that comprises, or competes for binding to domain 3 of human LAG-3 with an antibody comprising, the six CDRs of antibody 410C9; and
   a pharmaceutically acceptable carrier.
89. A method comprising administering an effective amount of the pharmaceutical composition of any one of embodiments 83 to 88 to an individual in need thereof.
90. A method of administering an anti-LAG-3 immunotherapy to an individual in need thereof, the method comprising administering to the individual an effective amount of the pharmaceutical composition of any one of embodiments 83 to 88.
91. The method according to embodiment 90, wherein the anti-LAG-3 immunotherapy is administered as a monotherapy.
92. The method according to embodiment 90, wherein the anti-LAG-3 immunotherapy does not comprise administering an agent that targets an immune checkpoint molecule other than LAG-3 to the individual.
93. The method according to embodiment 90, wherein the anti-LAG-3 immunotherapy does not comprise administering an anti-PD1 agent to the individual.
94. The method according to any one of embodiments 90 to 93, wherein the individual has cancer, and wherein the anti-LAG-3 immunotherapy is effective in treating the cancer.
95. The method according to embodiment 94, wherein the cancer comprises a solid tumor.
96. The method according to embodiment 95, wherein the cancer comprises a carcinoma, lymphoma, blastoma, or sarcoma, optionally wherein the cancer comprises non-small cell lung cancer (NSCLC), head and neck squamous cell carcinoma (HNSCC), renal cell carcinoma (RCC), breast cancer, triple-negative breast cancer (TNBC), or melanoma.
97. The method according to embodiment 94, wherein the cancer is a hematologic malignancy.
98. The method according to embodiment 97, wherein the hematologic malignancy is a leukemia, a lymphoma, or multiple myeloma.

The following examples are offered by way of illustration and not by way of limitation.

### EXPERIMENTAL

### Example 1—Crystal Structure of LAG-3 Dimer

Described in this example is the determination of the crystal structure of the mouse LAG-3 dimer.

For structural studies, the LAG-3 extracellular domain (ECD) was cloned into the gWIZ vector with a C-terminal 6×His tag. DNA was transiently transfected into Expi293F™ cells (ThermoFisher Scientific) using Expifectamine™ reagent (ThermoFisher Scientific). Five days post-transfection, supernatant was harvested, adjusted to pH 8.0, and sterile filtered. The mouse LAG-3 ECD was then purified using cobalt agarose resin (GoldBio) and concentrated in 25 mM Tris, 150 mM NaCl, pH 8.0 using Amicon Centrifugal Filters (Millipore Sigma, Burlington, MA).

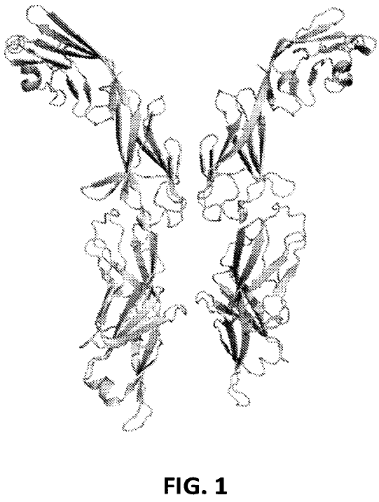

Shown in FIG. 1 is the crystal structure of LAG-3 as a dimer. Although sometimes X-ray crystallography shows proteins as dimers or multimers as an artifact of the crystallization process, FIGS. 1 and 3 herein indicate that LAG-3 is a dimer in its native state, and that dimerization is via domain 2 of LAG-3. Also observed in FIG. 1 is that LAG-3 is a dimer by size exclusion chromatography.

### Example 2—the C9B7W Anti-Mouse LAG-3 Antibody Binds Domain 2 of Mouse LAG-3 and Functions by Disrupting LAG-3 Dimerization

Domains 1 and 2 (D1D2) of the mouse LAG-3 ECD were cloned as a N-terminal fusion to Aga2 using a vector designated pPK which contains an Aga2p signal peptide and a cMyc tag between LAG-3 D1D2 and Aga2. Saccharomyces cerevisiae strain EBY100 was transformed with the pPK LAG-3 D1D2 vector and plated on SD-CAA plates for 3 days. Individual colonies were picked into SD-CAA liquid, grown overnight, and transferred to SG-CAA for protein induction. Yeast displaying LAG-3 D1D2 were incubated with respective antibodies to obtain a binding curve and estimated Kd.

For epitope mapping studies, plasmids encoding pPK LAG-3 D1D2 with single alanine mutants of solvent-accessible residues were ordered from Twist Biosciences or GenScript. Each individual plasmid was transformed into EBY100 using the aforementioned methods. In 96-well plate format, each alanine mutant was arrayed, and then incubated with antibodies at their approximate Kd (as measured by yeast surface display binding). The 96-well plates were then run on a BD Accuri to obtain hits that reduced antibody binding signal compared to WT pPK LAG-3 D1D2.

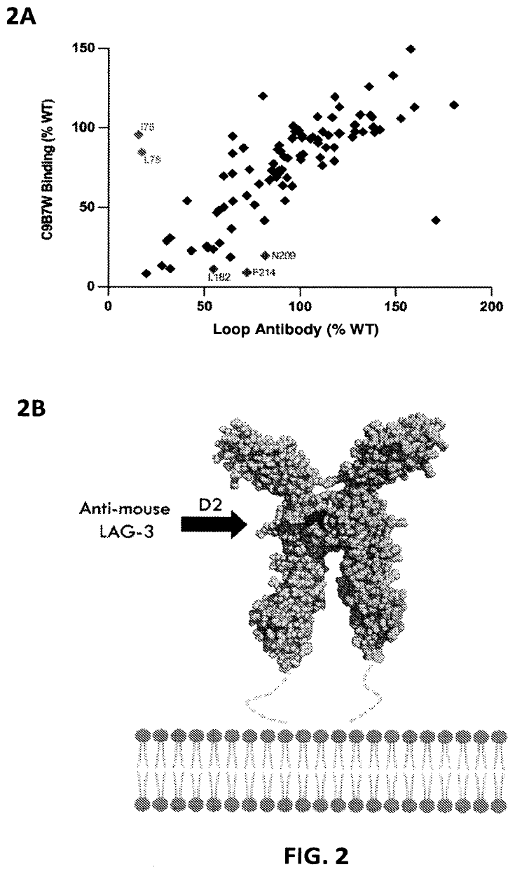

FIG. 2 provides data showing the percent of binding of each alanine mutant relative to binding of WT pPK LAG-3 D1D2 to C9B7W (anti-mouse LAG-3 antibody-binds D2) and M2-6-8 (D1 antibody). L182A, N209A, and F214A knocked out binding for C9B7W but not for M2-6-8, indicating that this is the binding epitope for C9B7W.

FIG. 3 provides electron micrographs revealing that the C9B7W anti-mouse LAG-3 antibody functions by blocking LAG-3 dimerization. 3A: electron micrographs showing LAG-3 in the absence of treatment with C9B7W. 3B: electron micrographs showing disruption of LAG-3 dimerization by treatment of LAG-3 with a Fab version of C9B7W.

### Example 3-Blocking LAG-3 Dimerization/Signaling and Maintaining MHCII Binding is Superior to Knocking Out LAG-3

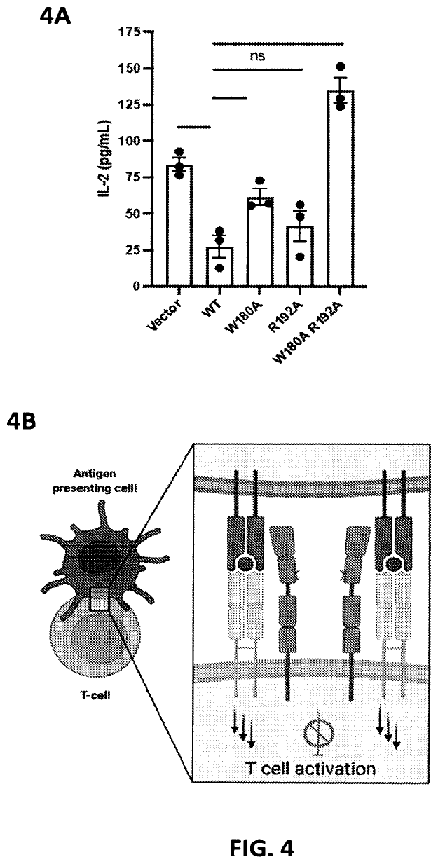

Shown in FIG. 4A is data indicating that introducing mutations at the dimer interface (W180A and R192A) to prevent LAG-3 dimerization (and in turn, LAG-3 signaling), but allowing LAG-3 to retain its binding to MHCII, results in a significant increase in IL-2 secretion compared to having no LAG-3 in the system. Typically, when LAG-3 is expressed on T cells, a decrease in IL-2 secretion is observed, but adding a dimerization disrupted/“signaling dead” version of LAG-3 greatly enhances IL-2 secretion. This is shown genetically in the present example, and it is reasonably expected from this data that a LAG-3 dimerization disrupting agent (e.g., a LAG-3 dimerization disrupting agent) would function through this mechanism pharmacologically. By bridging T cells with antigen-presenting cells, “signaling dead” LAG-3 is able to further enhance the immune synapse and drive IL-2 secretion. This data is further supported by data from Workman et al., Journal of Immunology 169(10):5392-5 (2002) that shows that LAG-3 without its intracellular domain is also capable of enhanced immune synapse formation as measured by increased IL-2 secretion in CD4 T cell hybridomas.

For LAG-3 and LAG-3 mutant construction, LAG-3 cDNA fragments were amplified by PCR and cloned into a pCDH lentiviral expression vector (SystemBio #CD710B-1) under the EF1α-HTLV promoter using Gibson assembly. For FRET related assays, LAG-3 fragments were cloned into pECFP and pEYFP plasmids. Alanine mutants were constructed by designing two complementary mutagenic primers and using N- and C-terminal flanking primers to amplify two individual fragments that were cloned into the expression vector using Gibson assembly.

For lentiviral transduction of T-cells, plasmids were reverse transfected into 293T cells to generate lentivirus. A pCDH:psPAX2:V-SVG DNA ratio of 2:1.5:1 and a Lipo2000:DNA ratio of 3:2 was used. After 16 hours, the media was removed and replaced with RPMI 1640 supplemented with 10% FBS. After an additional 24 hours, viral supernatant was collected for transduction of 3A9 (ATCC #CRL-3293). Transduced cells were selected with puromycin 48 hours after transduction, sorted for LAG-3+ cells, and used for subsequent functional assays. Cells were maintained in RPMI 1640 supplemented with 10% FBS.

For the IL-2 assay and quantitation, LK35.2 cells (ATCC #HB-98) were pulsed with 1 μM HEL peptide (MBL International #TS-M708-P) for 30 minutes at 37° C. 3A9 (ATCC #CRL-3293) cells were added in a 3A9:LK35.2 ratio of 4:1. After 24 hours, supernatant was collected and used for IL-2 quantitation by alphaLISA (Perkin Elmer #AL585C).

### Example 4—Both Mouse and Human LAG-3 are Capable of Dimerization

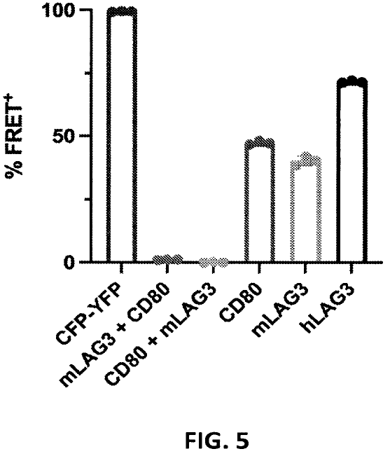

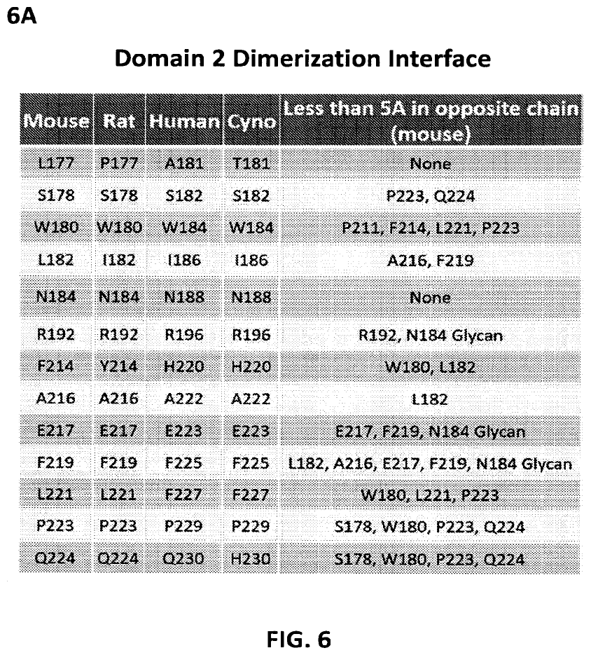

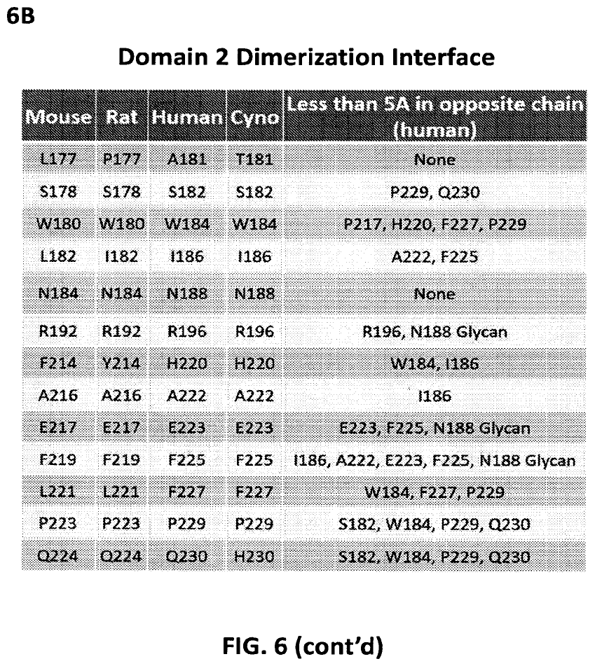

Shown in FIG. 5 is data demonstrating that mouse and human LAG-3 are both capable of dimerization. For the FRET assay, Expi293 cells (ThermoFisher) were transfected with a CFP:YFP:PEI ratio of 0.5:0.5:3 in a 24-well plate at 37° C. in 8% CO. Cells were analyzed after 36 hours using a BD LSRII flow cytometer. The 407 nm violet laser was used to excite CFP and the 488 nm blue laser was used to excite YFP. CFP was detected using a 450/50 bandpass filter after excitation with the violet laser and YFP was detected using a 530/30 bandpass filter after excitation with the blue laser. FRET was detected using the 525/50 bandpass and 505 longpass filter after excitation with the violet laser. A CFP-YFP genetic fusion and the known homodimer CD80 were used as positive controls. CD80 and mouse LAG-3 were also co-transfected as negative controls, since they are not known to dimerize. As shown by FRET in FIG. 5, CD80, mouse LAG-3, and human LAG-3 are all capable of homodimerization.

### Example 5—Structure of Glycosylated Mouse LAG-3 Extracellular Domain

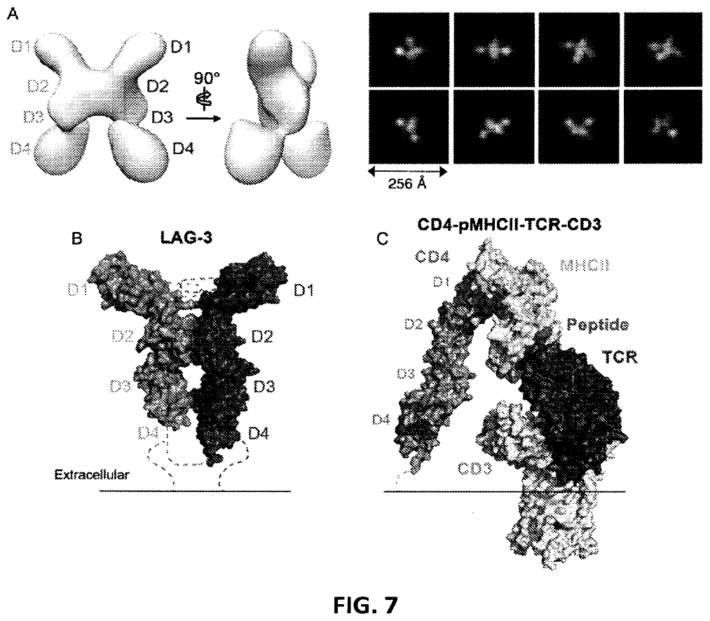

To elucidate the apo structure of glycosylated LAG-3, a soluble form of fully glycosylated mouse LAG-3 extracellular domain (ECD) was expressed and crystallized. Despite numerous attempts to fill the electron density from the X-ray scattering data, the structure was unable to be resolved. To achieve success, several methods were utilized, combining X-ray electron density datasets with negative stain electron microscopy (EM; FIG. 7A), and the AlphaFold predicted structure of human LAG-3, to ultimately resolve the 3D structure of glycosylated mouse LAG-3 protein to 3.82A (FIG. 7B).

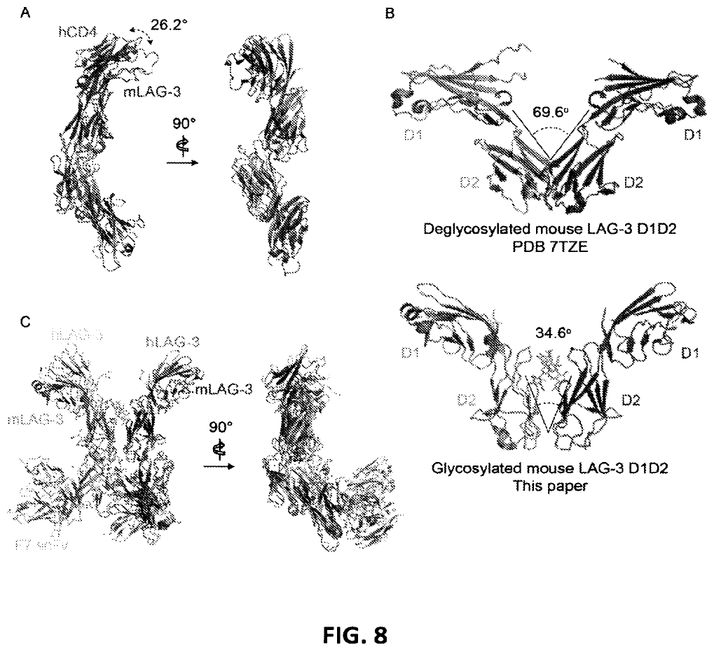

LAG-3's predicted height from the cell surface appears to be similar to that of the CD4:MHCII:TCR complex (FIG. 7C). LAG-3 D1 consists of an Ig-like V-type domain, followed by three Ig-like C2-type domains that make up D2 through D4, in the same orientation as CD4. Compared to the CD4 apo structure, LAG-3 D1 is rotated outwards approximately 26° (FIG. 8). This rotation allows additional space for an extra 30 amino acid loop in LAG-3 (Loop 1) to fit into the dimeric protein without clashing with the opposite chain of the LAG-3 dimer (FIG. 7C). Loop 1 has been shown to play a critical role in LAG-3 binding to MHCII, as its removal completely disrupts LAG-3 binding to MHCII (Huard PNAS 1997). In the structural model, Loop 1 could not be completely built due to its long, flexible nature; however, a portion of the human LAG-3 loop, in complex with an antibody fragment, has recently been resolved (BMS CCR 2022; PDB 7UM3).

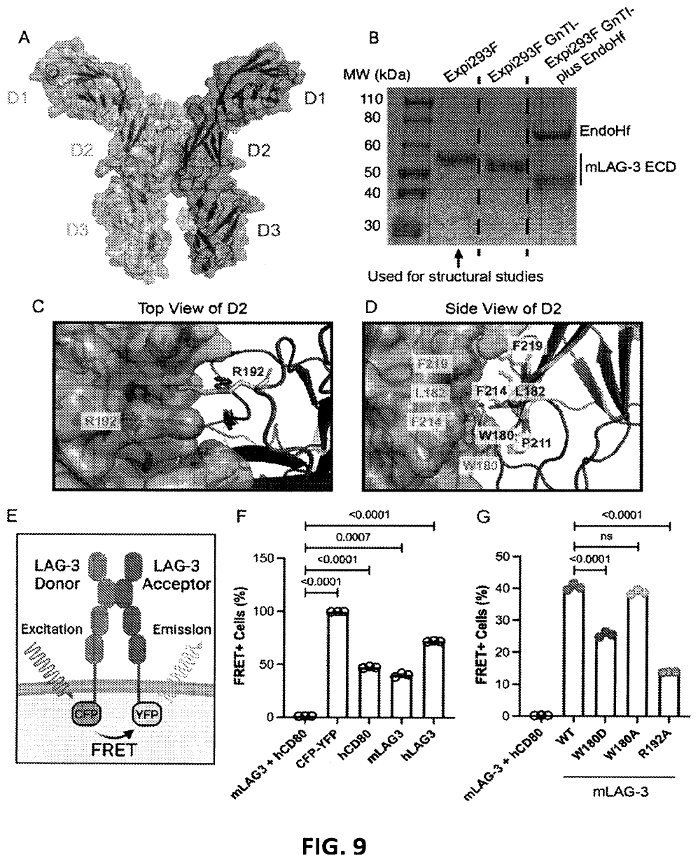

Although previous reports have suggested that LAG-3 dimerizes through the D1 domain (Huard PNAS, 1997; Li, JI 2004), the present structure confirms glycosylated LAG-3 dimerization through D2 (FIG. 9A). Upon removal of all N-linked glycans from LAG-3 by using Expi293F GnTI-cells and endoglycosidase EndoHf, a 10 kDa shift in protein size by SDS-PAGE was observed, indicating approximately 20 kDa of N-linked glycans on the dimeric protein FIG. 9B). By using glycosylated LAG-3 in the present study, it was possible to visualize two unique, bulky N-linked glycans significantly contributing to the dimerization interface, bridging an N-linked glycan on N184 with residue R192 on the opposite chain of the LAG3 dimer (FIG. 9C). This glycan-protein interaction dramatically changes the orientation of LAG-3 and its positioning for binding to pMHCII. In the absence of glycans, the D1 and D2 domains of LAG-3 present in a “winged” fashion, with an angle of ˜70° (FIG. 8B). With a glycan at N184, LAG-3 has a more upright “V” orientation, with a ˜35° angle (FIG. 8B). This orientation impacts the degree of contact between the extensive network of hydrophobic interactions directly below the glycan-protein interface.

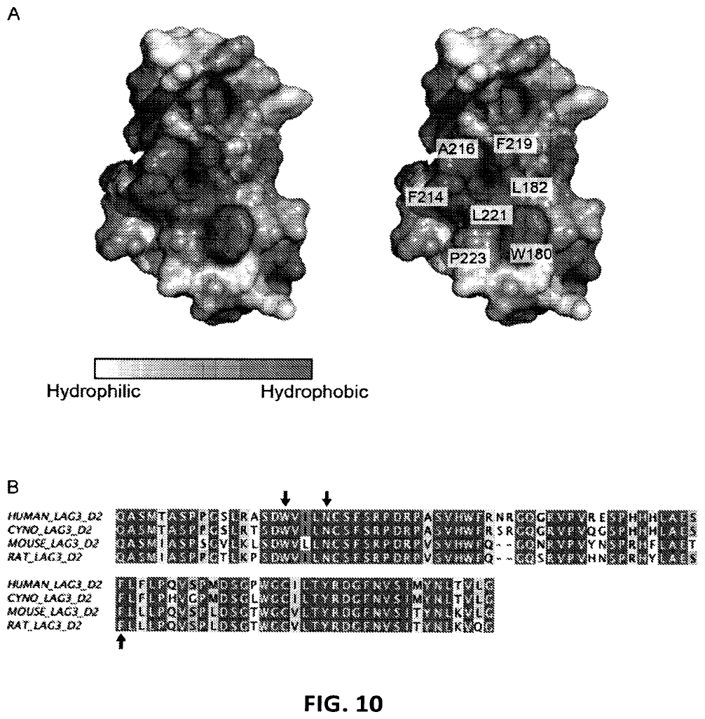

An evolutionarily conserved hydrophobic interface contributes the majority of the buried surface area to the D2-mediated cis-homodimerization (FIG. 10). It was possible to visualize protein-protein contacts between 7 hydrophobic residues from the D2 domain of each monomer, including W180, L182, F214, A216, F219, L221, and P223 (FIG. 9D, 10). These interactions allow the cis-homodimer to form in the absence of glycans (FIG. 8B); however, the buried surface area observed in the structures of deglycosylated mouse and human LAG-3 D2 is approximately 440 and 438 angstroms, respectively. The present glycosylated LAG-3 D2 buries 511 angstroms of surface area, potentially due to stabilization from the bulky glycan-protein interaction at positions N184 and R192 in the present structure (FIG. 8B). Furthermore, when comparing the present structure to the deglycosylated full-length human LAG-3 ECD, observed is a larger angle between the mouse LAG-3 D1 domains (23.9° vs) 34.6° and an outward rotation of the human LAG-3 D4 domain. These differences may potentially be explained by variances in glycosylation state, crystal packing, mutation of M171 to isoleucine in the human LAG-3 protein, or because the prior structure represented a co-complex of human LAG-3 with a D4-binding scFv.

While the present studies confirm that the extracellular domain of LAG-3 is a dimer, it was unclear whether full-length LAG-3 also dimerizes. Flow cytometry-based Förster resonance energy transfer (flow-FRET) assays have been utilized for live cell detection of proximal protein associations at distances <100 Å and was thus used to assess LAG-3 dimerization. To analyze LAG-3 on the cell surface. Expi293F cells were co-transfected with both LAG-3 CFP and LAG-3 YFP and compared against other fusion proteins for detection of FRET (FIG. 9E). Indeed, both mouse and human LAG-3 showed a positive FRET signal, suggesting dimerization on the surface of cells (FIG. 9F). LAG-3 co-transfected with CD80 was used as a negative control, as there has been no reported association between the two proteins. Indeed, when mouse LAG-3 CFP was co-transfected with CD80 YFP, there was no FRET signal, suggesting no association between these two receptors (FIG. 9F).

Next, single point mutations were introduced within the D2 dimerization interface to assess whether dimer formation could be disrupted and measured by FRET. Mutation of W180 to aspartic acid in LAG-3 Chain A (CFP-tagged) and Chain B (YFP-tagged) led to a significant reduction in FRET signal, indicating a reduction in dimerization (FIG. 9G). For the chains to repel each other, it was necessary to introduce a charged residue into the hydrophobic interface, as mutation to a minimal hydrophobic residue using W180A had no effect on LAG-3 FRET signal (FIG. 9G). A reduction in FRET signal for R192A, which mediates the key glycan-protein interaction at the dimer interface, was also observed; however, it was decided to not perform additional studies with this mutation due the ability of mouse LAG-3 to still maintain a cis-homodimer in the absence of the glycan-protein interaction (FIG. 8B).

### Example 6—LAG-3 Cis-Homodimerization is Required for MHCII Binding

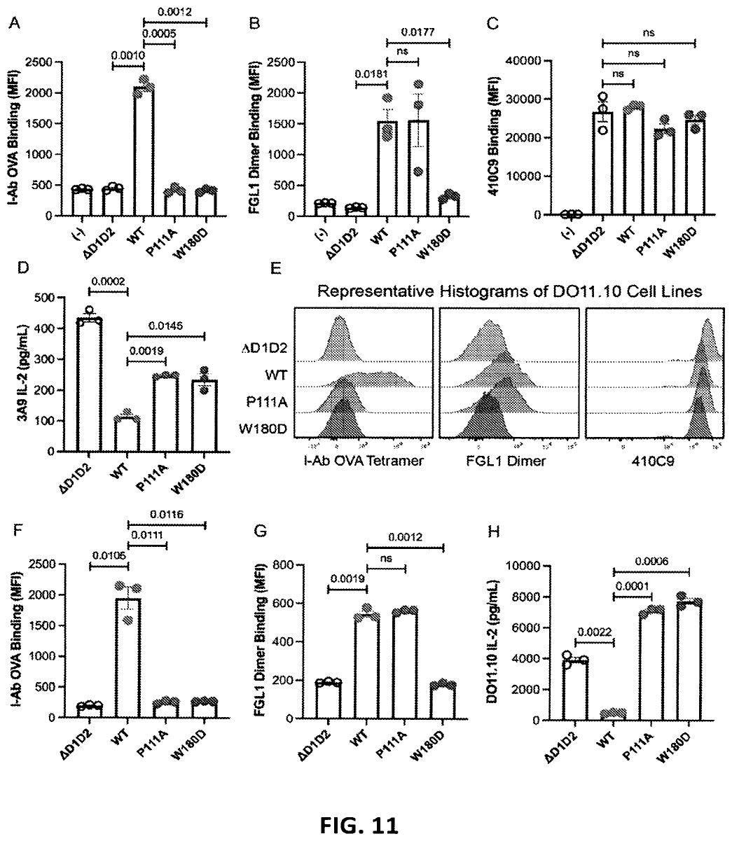

It was then sought to determine whether a reduction in dimerization impacted LAG-3 binding to MHC-II as well as LAG-3 function. To this end, the dimerization-affecting mutant W180D was stably expressed in the CD4⁺I-A^{k}-HEL₅₀₋₆₂ reactive T cell hybridoma, 3A9. Employed was a tetrameric form of MHC-II (I-A^{b}) covalently bound to ovalbumin peptide (OVA₃₂₃₋₃₃₉), a non-cognate peptide, in order to selectively bind LAG-3. Also tested was a truncated form of LAG-3 containing only domains 3 and 4 of LAG-3 (ΔD1D2) and the previously characterized D1 mutation P111A, which cannot bind pMHCII (Maruhashi 2018). The dimer-disrupting mutation W180D completely nullified 3A9 binding to pMHCII tetramer, to levels comparable to ΔD1D2 and P111A (FIG. 11A). Tested next was these cells' ability to bind to LAG-3's non-canonical ligand, FGL1. While P111A-expressing 3A9 cells had equal levels of binding as WT mouse LAG-3, ΔD1D2 and W180D both had significantly reduced capacity to bind FGL1 (FIG. 11B). Both P111A and W180D showed equivalent expression to controls, as measured by an antibody against LAG-3 D3 (410C9). Evaluated next was whether these LAG-3 mutants could inhibit T cells. When stimulated by LK35.2 cells presenting I-A^{k}-HEL₅₀₋₆₂ peptide, both P111A and W180D mutations relieved LAG-3 inhibition, as observed by elevated IL-2 production (FIG. 11D). To further confirm these results and the impact of W180D on LAG-3 function, these experiments were repeated in the DO11.10 T cell hybridoma (FIG. 11E-H). Consistent with the 3A9 findings, the W180D mutation in DO11.10 T cells also abrogated LAG-3's ability to bind pMHCII and FGL1 (FIG. 11F-G). This inability to bind to its ligands also resulted in reduced LAG-3 function in the context of cognate pMHCII recognition by TCR (FIG. 11H). These data indicate that cis-homodimerization is key to LAG-3's ability to bind to pMHCII and FGL1, and that disruption of this cis-homodimerization results in reduced LAG-3 function.

### Example 7—Mouse LAG-3 Antibody C9B7W Binds at the Dimer Interface

With newfound interest in the D2 domain and its function in LAG-3 biology, sought next was to understand the binding epitope of a commonly used mouse LAG-3 specific, D2-binding antibody, C9B7W, which is capable of blocking MHCII-mediated LAG-3 signaling. Although C9B7W is capable of blocking MHCII-dependent LAG-3 signaling, debate has surrounded the antibody's ability to block MHCII binding to LAG-3 (Workman 2002, Cemerski 2015, Maeda, 2019, Everett 2019, Maruhashi 2022). Guided by the present structure of mouse LAG-3, fine epitope mapping of C9B7W was performed. A library of individual alanine mutations of solvent accessible residues within the D1 and D2 domains was created. These 95 mutant LAG-3 D1D2 proteins, along with the wild-type LAG-3 D1D2 domains, were individually displayed on the surface of yeast, fused to the Aga2p mating protein (FIG. 12). Using flow cytometry and fluorescent antibody staining, LAG-3 variant protein expression levels (using an antibody against a Myc tag between LAG-3 and Aga2p) and binding to C9B7W were then measured. Also used was an in-house Loop 1 antibody (M2-6-8) as a control to measure protein folding and expression.

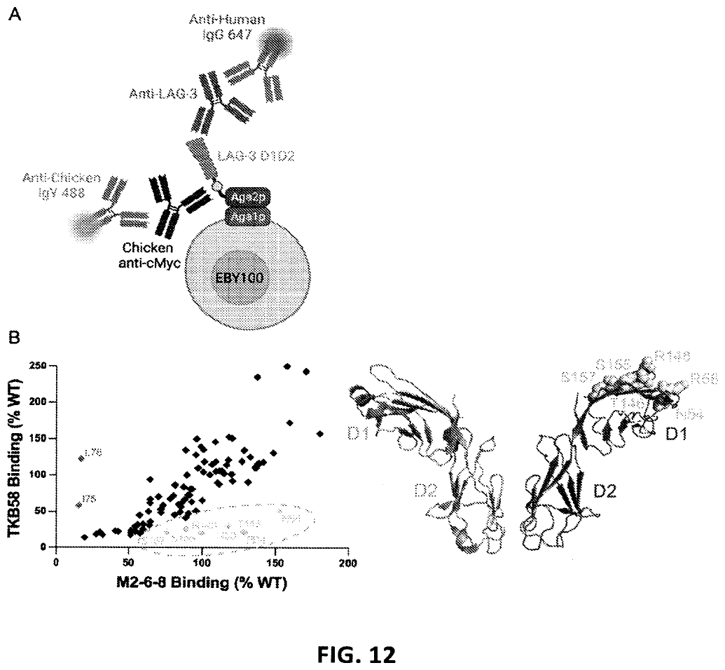

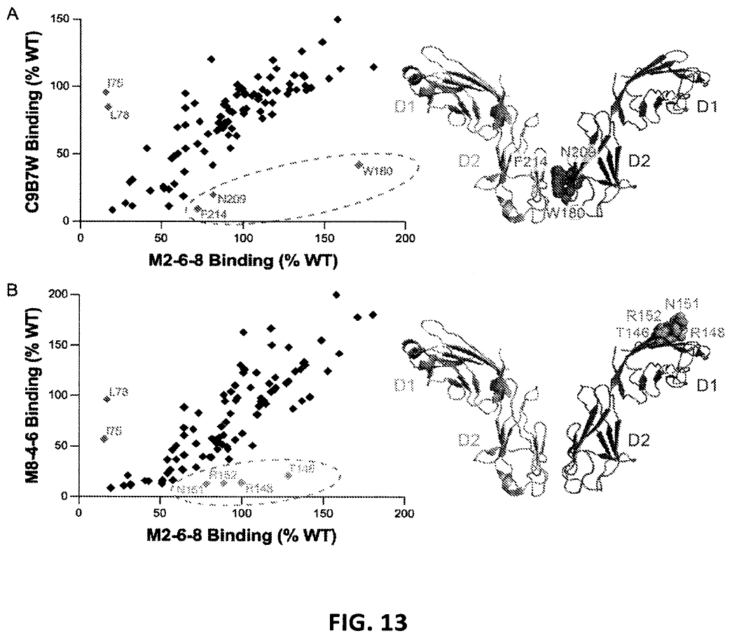

Using this method of fine epitope mapping, it was determined that C9B7W binds at the LAG-3 D2 dimer interface (FIG. 13A). Specifically, LAG-3 mutations of W180, N209, F214, and E217 to alanine resulted in reduced binding signal for C9B7W compared to M2-6-8 (FIG. 13A). The measured affinity for C9B7W using monomeric D1D2 on the surface of yeast (136 pM) was significantly different than a previous report using soluble dimeric LAG-3, which estimated a K_{D} of 413.9 nM (Maeda, J B C 2019). This difference is perhaps due to the competition of C9B7W with LAG-3 self-association in the context of dimeric LAG-3.

### Example 8—Antibodies Against the Tip of D1 can Potently Block MHCII and FGL1 Binding

With the alanine library in hand, sought next was to epitope map additional antibodies that are capable of potently disrupting LAG-3's interaction with pMHCII and FGL1. Using LAG3 KO mice, a set of antibodies against LAG-3 with various specificities was generated. Identified was an antibody, M8-4-6, that was capable of binding to D1, blocking the LAG-3:pMHCII interaction, and blocking the LAG-3:FGL1 interaction, in a manner similar to TK58. This antibody was epitope mapped to the tip of D1. In particular, mutation of residues T146. R148, N151, and R152 to alanine resulted in a dramatic decrease in M8-4-6 binding signal compared to M2-6-8. (FIG. 13B). This region appears to be on the opposite face of D1 from Loop 1 (MHCII binding site) and Loop 2. To gain additional insights into D1-binding, pMHCII/FGL1 dual-blocking antibodies. TKB58 (Okazaki Immunity 2022) was also epitope mapped. This antibody also bound at the tip of D1, with an overlapping epitope to M8-4-6 (FIG. 12). The analysis revealed that TKB58 shares binding contacts with T148 and R148, while also binding to residues N54, R58, R94, S155, and S157, as suggested by alanine mutagenesis. Despite their binding at a distinct site away from Loops 1 and 2, M8-4-6 and TKB58 are both able to potently block LAG-3 binding to pMHCII and FGL1 (Okazaki Immunity 2022).

### Example 9—Antibodies Against LAG-3 can Disrupt LAG-3 Dimerization

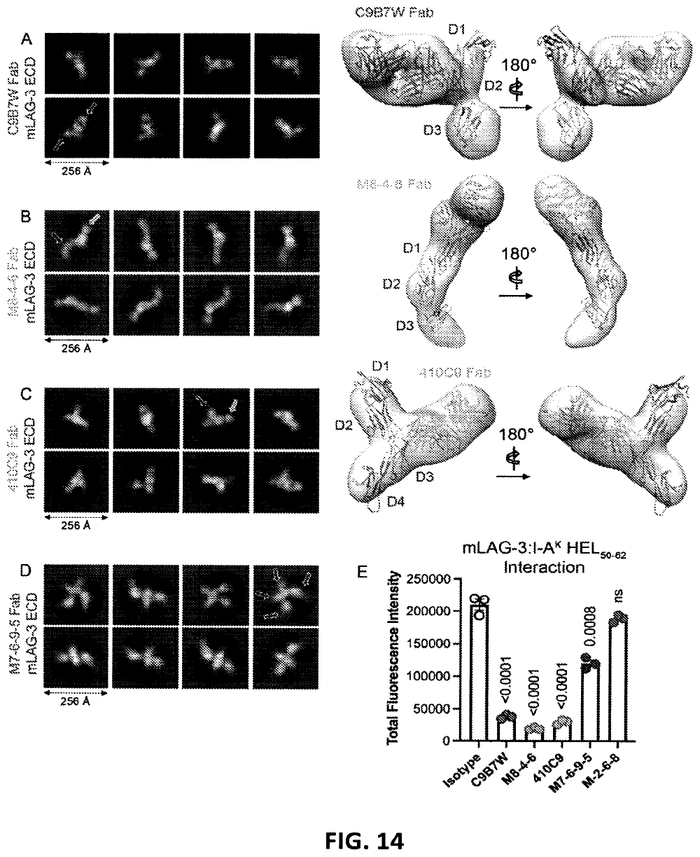

Following the results of the above-described C9B7W epitope mapping experiments proposing that this antibody binds to the D2 interface, tested next was whether C9B7W was capable of disrupting LAG-3 dimerization, building on the initial negative stain EM studies (FIG. 7A). A Fab fragment of C9B7W was prepared and incubated with mouse LAG-3 in a 1:1 ratio, and the resulting complex placed onto grids for negative stain EM. While apo mouse LAG-3 formed dimers on the grid (FIG. 7A, 8), mouse LAG-3 complexed with C9B7W was only present in monomeric form (FIG. 14A). The present structure, negative stain EM, and epitope mapping data was used to reconstruct a 3D model of C9B7W bound to mouse LAG-3 (FIG. 14A). These data, combined with the finding that the dimer-disrupting mutant W180D is incapable of binding to pMHCII, support the hypothesis that C9B7W can activate T cells by blocking LAG-3 dimerization-dependent binding to pMHCII.

Since M8-4-6 is also capable of disrupting LAG-3 binding to MHCII and FGL1, despite binding at a distinct site away from Loops 1 and 2, tested was whether M8-4-6 was also capable of disrupting LAG-3 cis-homodimerization. When the M8-4-6 Fab was co-complexed with mouse LAG-3 and analyzed by negative stain EM, it was also capable of disrupting the LAG-3 dimer (FIG. 14B), even though this antibody appears to bind to the tip of the LAG-3 D1 domain. This finding may explain why M8-4-6 (and by extension TKB58) is able to block binding of both MHCII and FGL1 ligands, despite engagement via epitopes distinct from the ligands' purported binding sites on LAG-3.

During screening for LAG-3 antibodies capable of disrupting LAG-3 binding to pMHCII and FGL1, also identified was a commonly used antibody, 410C9, known to block LAG-3 binding to both ligands (Mao et al. Science 2016). This finding was surprising, given 410C9's binding to D3, a site more than 30 Å away from D1. Tested was whether this antibody was also capable of disrupting LAG-3 dimerization. When co-complexed with mouse LAG-3, the 410C9 Fab fragment was also shown to disrupt LAG-3 dimerization by EM analysis (FIG. 14C). Since it was not feasible to epitope map this D3-binding antibody with the yeast surface-displayed D1-D2 alanine mutant library, AlphaFold-based prediction of 410C9 binding to the D3 domain was used to assist in positioning the Fab within the electron density map (FIG. 14C). In contrast to 410C9, another in-house generated antibody against the D3 domain of mouse LAG-3 (M7-6-9-5) was not able to disrupt LAG-3 dimerization and was clearly visualized in complex with intact LAG-3 dimer on EM grids (FIG. 14D). Consistent with this finding, the recently solved structure of human LAG-3 in complex with a human D4-binding antibody (F7) shows that LAG-3 can dimerize in the context of certain antibodies.

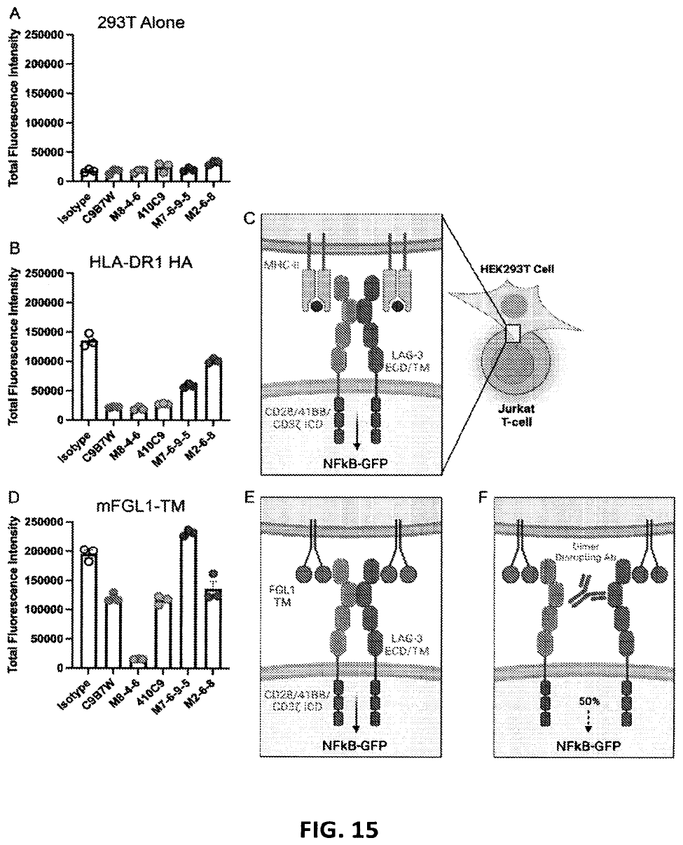

Finally, it was desirable to test these antibodies' ability to block pMHCII binding in a sensitive, in-house developed LAG-3 chimeric antigen receptor (CAR) assay (FIG. 15). In this assay, 293T cells stably expressing pMHCII are co-cultured with 3A9 cells stably expressing a NFkB-GFP reporter and a LAG-3 CAR. This chimeric protein consists of the LAG-3 ECD, followed by CD28/41BB/CD3z ICD. Upon ligand engagement by the LAG-3 CAR, the 3A9 cells become activated, which results in NFkB-dependent GFP expression. This assay can be used to test LAG-3 blocking antibodies in a high throughput, sensitive manner. Upon adding C9B7W (anti-D2), M8-4-6 (anti-D1), or 410C9 (anti-D3) to the co-culture of 293T cells expressing I-A^{k}-HEL₅₀-62, all three potently block LAG-3-mediated CAR signaling (FIG. 14E). In contrast, the non-dimer disrupting M7-6-9-5 antibody is unable to block LAG-3-mediated signaling. Similarly, the Loop 1-binding M2-6-8 is also incapable of blocking in this assay; however, this may be due to its specific epitope on Loop 1, as other Loop 1-binding antibodies have been shown to block the LAG-3:pMHCII interaction (Relatlimab:loop structure paper). Altogether, these data demonstrate that antibodies against LAG-3 D1, D2, or D3 can block LAG-3 dimerization and ligand binding.

### SUMMARY

LAG-3 regulates T cell responses in the context of cancer and autoimmunity. However, the molecular mechanism and functional relevance of LAG-3 dimerization is currently unknown. In the studies above, the structure of the glycosylated LAG-3 homodimer was solved and the importance of glycosylation and cis-homodimerization in LAG-3 binding to pMHCII was identified. Using epitope mapping, it was shown that the antibody C9B7W is able to disrupt LAG-3 dimerization, which has implications for its use as a therapeutic agent.

The present structural analysis of the LAG-3 dimer revealed that two LAG-3 monomers interact through an extended interface, which mainly consists of the IgC D2 domain. This interface is stabilized by hydrogen bonds and hydrophobic interactions, which are likely important for the formation of the stable dimer. Furthermore, it was identified that positioning of D1 by means of D2 homodimerization is dependent on the presence of N-linked glycans on N184. This suggests that glycosylation plays a crucial role in the formation and stability of the LAG-3 homodimer.

The present findings on the importance of LAG-3 glycosylation and homodimerization in positioning D1 led to investigation of the potential of disrupting the LAG-3 dimer interface as a means of modulating its activity. To this end, mutations at key residues in the dimer interface were created and their effects on flow-FRET-based dimerization and MHCII binding were characterized. Despite its ability to disrupt dimerization in our flow-FRET-based assay, R192A was deprioritized for further study when analyzing the deglycosylated mouse LAG-3 D1D2 structure. W180 was identified as an interface residue with the most interchain contacts and a hydrophobic-to-charge mutation was used to disrupt the LAG-3 dimer. This mutated LAG-3 W180D showed reduced binding to ligands, as well as reduced LAG-3 function.

The antibody C9B7W was also epitope mapped to show that it can disrupt LAG-3 dimerization, which may have therapeutic implications for the treatment of cancer and autoimmune diseases. This antibody targets a hydrophobic patch in the LAG-3 IgC domain D2, specifically at the dimerization interface. The data suggest that C9B7W interferes with the formation of LAG-3 homodimer by binding to the IgC domain of one of the monomers, which prevents the formation of the dimer. Although the study focused on MHCII, homodimerization may also play a role in LAG-3 binding to other ligands, as C9B7W has been shown to block the LSECtin:LAG-3 and a-Synuclein:LAG-3 interactions.

Finally, the present results also indicate that other antibodies against D1 and D3 of LAG-3 are capable of disrupting LAG-3 dimerization, implicating these domains as targets for the development of therapeutics. This disruption appears to be epitope-dependent, as the D3-binding 410C9 can disrupt LAG-3 dimerization while M7-6-9-5 cannot.

In conclusion, the present study demonstrates that LAG-3 homodimerization and glycosylation are critical for the formation of stable LAG-3 homodimer and its binding to MHCII. The findings provide a structural basis for the identification of therapeutics that target LAG-3 dimerization, with implications for the treatment of cancer, autoimmune disease, etc.

### Materials and Methods

### Protein Production and Purification

For structural studies, the LAG3 ECD was cloned into the gWIZ vector with a C-terminal 6×His tag. DNA was transiently transfected into Expi293F cells (ThermoFisher Scientific) using Expifectamine reagent (ThermoFisher Scientific). Five days post-transfection, supernatant was harvested, adjusted to pH 8.0, and sterile filtered. LAG3 ECD was then purified using cobalt agarose resin (GoldBio) and concentrated in 25 mM Tris, 150 mM NaCl, pH 8.0 using Amicon Centrifugal Filters (Millipore Sigma. Burlington, MA).

### X-Ray Crystallography Sample Preparation, Data Collection, and Structure Determination

LAG3 ECD was concentrated to 7 mg/mL and mixed with 0.15 uL of 0.1 M Tris, 0.05 M L-Arginine, 0.05 M L-Glutamic acid monosodium salt hydrate, 14% PEG4000 at pH 8.1 in a 1:1 ratio. Crystals were grown by sitting drop vapor diffusion at 16° C. Cryoprotection, diffraction and structure determination from Mathews.

### LAG3 and LAG3 Mutant Construction

LAG3 cDNA fragments were amplified by PCR and cloned into a pCDH lentiviral expression vector (SystemBio #CD710B-1) under the EF1α-HTLV promoter using Gibson assembly. For FRET related assays, LAG3 fragments were cloned into pECFP and pEYFP plasmids (provided by Dr. Xiangpeng Kong). Mutants were constructed by designing two complementary mutagenic primers and using N and C-terminal flanking primers to amplify two individual fragments that were cloned into the expression vector using Gibson assembly.

### Flow Cytometric FRET

Expi293F cells (ThermoFisher Scientific) were transfected with a CFP:YFP:PEI ratio of 0.5:0.5:3 in a 24-well plate at 37° C. in 8% CO₂. Cells were analyzed after 36 hours using a BD LSRII flow cytometer. The 407 nm violet laser was used to excite CFP and the 488 nm blue laser was used to excite YFP. CFP was detected using a 450/50 bandpass filter after excitation with the violet laser and YFP was detected using a 530/30 bandpass filter after excitation with the blue laser. FRET was detected using the 525/50 bandpass and 505 longpass filter after excitation with the violet laser.

### Lentiviral Transduction of T-Cells

Plasmids were reverse transfected into 293T cells to generate lentivirus. A pCDH:psPAX2:V-SVG DNA ratio of 2:1.5:1 and a Lipo2000:DNA ratio of 3:2 was used. After 16 hours, the media was removed and replaced with RPMI 1640 supplemented with 10% FBS. After an additional 24 hours, viral supernatant was collected for transduction of 3A9 (ATCC #CRL-3293). Transduced cells were selected with puromycin 48 hours after transduction, sorted for LAG3+ cells, and used for subsequent functional assays. Cells were maintained in RPMI 1640 supplemented with 10% FBS.

### IL-2 Assay and Quantitation

LK35.2 cells (ATCC #HB-98) were pulsed with 1 μM HEL peptide (MBL International #TS-M708-P) for 30 minutes at 37° C. For antibody blocking experiments, 3A9 cells were incubated with either LAG3 blocking antibodies or an isotype control for 30 minutes at 37° C. 3A9 (ATCC #CRL-3293) cells were added in a 3A9:LK35.2 ratio of 4:1. After 24 hours, supernatant was collected and used for IL-2 quantitation by alphaLISA (Perkin Elmer #AL585C).

### Negative Stain EM Imaging, Data Processing and Structural Modeling

For negative stain EM, a 3 μl aliquot of the complex sample at a concentration of 0.01 μg/μL was applied onto a glow-discharged carbon-coated grid (FCF400-CU, Electron Microscopy Sciences, EMS), blotted with filter paper (Whatman 1), and stained using 0.75% (w/v) uranyl formate (EMS) for 30 s. The images were collected on FEI Talos L120C TEM at 120 kV coupled with a Gatan OneView camera. Each image was acquired in a low-dose mode at magnification of 73,000× resulting in a pixel size of 2.0 Å, using a dose rate of ˜40 e⁻/Å². All single particle processing including particle picking, 2D classification and 3D reconstruction was performed using cryoSPARC v3.3.0 (11).

### Yeast Surface Display

Domains 1 and 2 (D1D2) of LAG3 ECD were cloned as a N-terminal fusion to Aga2 using an in-house developed vector called pPK which contains Aga2p signal peptide and a cMyc tag between LAG3 D1D2 and Aga2. Saccharomyces cerevisiae strain EBY100 was transformed with the pPK LAG3 D1D2 vector and plated on SD-CAA plates for 3 days. Individual colonies were picked into SD-CAA liquid, grown overnight, and transferred to SG-CAA for protein induction. Yeast displaying LAG3 D1D2 were incubated with respective antibodies to obtain a binding curve and estimated Kd. For epitope mapping studies, yeast were incubated with antibodies at their approximate Kd (as measured by yeast surface display binding).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of generating cells that produce monoclonal antibodies that specifically bind domain 2 of a human lymphocyte activation gene-3 (LAG-3) polypeptide, the method comprising:
immunizing a non-human animal with an immunogen comprising domain 2 of a human LAG-3 polypeptide;
isolating monoclonal antibody-producing cells from the non-human animal, wherein the monoclonal antibody-producing cells produce monoclonal antibodies that specifically bind the immunogen; and
screening the monoclonal antibody-producing cells for cells that produce monoclonal antibodies that specifically bind domain 2 of the human LAG-3 polypeptide.

2. The method according to claim 1, wherein the immunogen consists of the extracellular domain of a human LAG-3 polypeptide or a fragment thereof comprising domain 2.

3. The method according to claim 1 or claim 2, wherein the immunogen consists of domains 1 and 2 of a human LAG-3 polypeptide or a fragment thereof comprising domain 2.

4. The method according to any one of claims 1 to 3, wherein the immunizing comprises administering the immunogen to the non-human animal.

5. The method according to any one of claims 1 to 3, wherein the immunizing comprises administering a nucleic acid encoding the immunogen to the non-human animal, wherein the nucleic acid is configured to express the immunogen upon administration to the non-human animal.

6. The method according to any one of claims 1 to 3, wherein the immunizing comprises administering cells that express the immunogen to the non-human animal, wherein the cells secrete the immunogen and/or display the immunogen on their surface upon administration to the non-human animal.

7. The method according to any one of claims 1 to 6, wherein the screening comprises identifying, among the monoclonal antibody-producing cells, cells that produce monoclonal antibodies that bind to a human LAG-3 polypeptide consisting of domain 2 or a fragment thereof.

8. The method according to claim 7, wherein the screening comprises identifying, among the monoclonal antibody-producing cells, cells that produce monoclonal antibodies that bind to a human LAG-3 polypeptide consisting of amino acids 175 to 230 of the human LAG-3 polypeptide set forth in SEQ ID NO:1 or a fragment thereof comprising amino acids I186, E215 and H220, wherein numbering is according to SEQ ID NO:1.

9. The method according to any one of claims 1 to 8, wherein the screening comprises a negative selection procedure to eliminate cells that produce monoclonal antibodies that bind to a domain of the human LAG-3 polypeptide other than domain 2.

10. The method according to any one of claims 1 to 8, wherein the screening comprises a negative selection procedure to eliminate cells that produce monoclonal antibodies that bind to a region of the human LAG-3 polypeptide other than a region within amino acids 175 to 230 of the human LAG-3 polypeptide set forth in SEQ ID NO:1.

11. The method according to any one of claims 1 to 10, wherein the screening comprises performing single cell sequencing on the monoclonal antibody-producing cells, determining the sequences of the antibodies produced by the monoclonal antibody-producing cells, and identifying cells as candidate cells that produce monoclonal antibodies that specifically bind domain 2 of the human LAG-3 polypeptide based on the sequences.

12. The method according to any one of claims 1 to 11, wherein the non-human animal is selected from the group consisting of: a mouse, a rat, a chicken, a cow, a goat, a llama, an alpaca, a shark, and a rabbit.

13. The method according to any one of claims 1 to 12, wherein the non-human animal comprises a replacement of one or more endogenous non-human animal immunoglobulin (Ig) genes with human Ig genes.

14. The method according to claim 13, wherein the monoclonal antibody-producing cells produce human/non-human animal chimeric monoclonal antibodies or fully human monoclonal antibodies that specifically bind the immunogen.

15. The method according to any one of claims 1 to 13, wherein the method further comprises screening monoclonal antibodies identified as specifically binding domain 2 of the human LAG-3 polypeptide for the ability to disrupt human LAG-3 dimerization.

16. The method according to claim 15, further comprising identifying a monoclonal antibody that disrupts LAG-3 dimerization as a LAG-3 dimerization disrupting agent.

17. The method according to any one of claims 1 to 16, further comprising immortalizing cells that produce monoclonal antibodies that specifically bind domain 2 of the human LAG-3 polypeptide.

18. A cell that produces a monoclonal antibody that specifically binds domain 2 of a human LAG-3 polypeptide obtained according to the method of any one of claims 1 to 17.

19. The cell of claim 18, wherein the monoclonal antibody specifically binds a human LAG-3 polypeptide within amino acids 175 to 230 of the human LAG-3 polypeptide set forth in SEQ ID NO: 1.

20. A monoclonal antibody comprising the six complementarity determining regions (CDRs) of a monoclonal antibody produced by the cell of claim 18 or claim 19.

21. A method of identifying a polypeptide that specifically binds domain 2 of a human LAG-3 polypeptide, the method comprising:
contacting a plurality of human LAG-3 polypeptides with a polypeptide library, wherein each of the plurality of human LAG-3 polypeptides comprise domain 2 of human LAG-3;
identifying polypeptides of the polypeptide library that specifically bind to human LAG-3 polypeptides of the plurality of human LAG-3 polypeptides; and
screening the polypeptides that bind to the human LAG-3 polypeptides for a monoclonal antibody that specifically binds domain 2 of human LAG-3.

22. The method according to claim 21, wherein the human LAG-3 polypeptides of the plurality are displayed on the surface of cells.

23. The method according to claim 22, wherein the cells are mammalian cells.

24. The method according to claim 22, wherein the cells are yeast cells.

25. The method according to claim 21, wherein the human LAG-3 polypeptides of the plurality are displayed on one or more solid supports.

26. The method according to claim 25, wherein the human LAG-3 polypeptides of the plurality are displayed on the surface of one or more wells.

27. The method according to claim 25, wherein the one or more solid supports comprise particulate solid supports.

28. The method according to claim 27, wherein the particulate solid supports comprise beads.

29. The method according to claim 21, wherein the human LAG-3 polypeptides of the plurality are complexed with ribosome-mRNA (RM) complexes during the contacting, and wherein the mRNAs encode the human LAG-3 polypeptides.

30. The method according to claim 21, wherein the human LAG-3 polypeptides of the plurality are complexed with DNAs during the contacting, and wherein the DNAs encode the human LAG-3 polypeptides.

31. The method according to claim 21, wherein the human LAG-3 polypeptides of the plurality are soluble during the contacting step.

32. The method according to any one of claims 21 to 31, wherein the polypeptides of the polypeptide library are displayed on the surface of phage during the contacting.

33. The method according to any one of claims 21 to 31, wherein the polypeptides of the polypeptide library are displayed on the surface of cells during the contacting.

34. The method according to claim 33, wherein the cells are mammalian cells.

35. The method according to claim 33, wherein the cells are yeast cells.

36. The method according to any one of claims 21 to 31, wherein the polypeptides of the polypeptide library are displayed on one or more solid supports.

37. The method according to claim 36, wherein the polypeptides of the polypeptide library are displayed on the surface of one or more wells.

38. The method according to claim 36, wherein the one or more solid supports comprise particulate solid supports.

39. The method according to claim 38, wherein the particulate solid supports comprise beads.

40. The method according to claim 38 or claim 39, wherein the particulate solid supports are magnetically responsive.

41. The method according to any one of claims 21 to 31, wherein the polypeptides of the polypeptide library are complexed with ribosome-mRNA (RM) complexes during the contacting, and wherein the mRNAs encode the polypeptides of the polypeptide library.

42. The method according to any one of claims 21 to 31, wherein the polypeptides of the polypeptide library are complexed with DNAs during the contacting, and wherein the DNAs encode the polypeptides of the polypeptide library.

43. The method according to any one of claims 21 to 31, wherein the polypeptides of the polypeptide library are soluble during the contacting step.

44. The method according to any one of claims 21 to 43, wherein each of the plurality of human LAG-3 polypeptides consist of the extracellular domain of a human LAG-3 polypeptide or a fragment thereof comprising domain 2.

45. The method according to any one of claims 21 to 43, wherein each of the plurality of human LAG-3 polypeptides consist of domains 1 and 2 of a human LAG-3 polypeptide or a fragment thereof comprising domain 2.

46. The method according to any one of claims 21 to 45, wherein the screening comprises identifying, among the identified polypeptides that bind to the human LAG-3 polypeptides, polypeptides that bind to a human LAG-3 polypeptide consisting of amino acids 175 to 230 of the human LAG-3 polypeptide set forth in SEQ ID NO:1 or a fragment thereof comprising amino acids I186, E215 and H220, wherein numbering is according to SEQ ID NO:1.

47. The method according to any one of claims 21 to 46, wherein the screening comprises a negative selection procedure to eliminate polypeptides that bind to a domain of the human LAG-3 polypeptide other than domain 2.

48. The method according to any one of claims 21 to 46, wherein the screening comprises a negative selection procedure to eliminate polypeptides that bind to a region of the human LAG-3 polypeptide other than a region within amino acids 175 to 230 of the human LAG-3 polypeptide set forth in SEQ ID NO:1.

49. The method according to any one of claims 21 to 48, wherein the polypeptide library is an antibody library.

50. The method according to claim 49, wherein the antibody library is a IgG library, a Fab library, an scFv library, or single variable domain located on a heavy chain (VHH) library.

51. The method according to claim 49 or claim 50, wherein the antibody library is a human chimeric or fully human antibody library.

52. The method according to any one of claims 21 to 48, wherein the polypeptide library is a knottin library, a fibronectin type-III (FNIII) domain library, or a designed ankyrin repeat protein (DARPin) library.

53. The method according to any one of claims 21 to 51, wherein the method further comprises screening polypeptides identified as specifically binding domain 2 of the human LAG-3 polypeptide for the ability to disrupt human LAG-3 dimerization.

54. The method according to claim 53, further comprising identifying a polypeptide that disrupts LAG-3 dimerization as a LAG-3 dimerization disrupting agent.

55. A polypeptide that specifically binds domain 2 of a human LAG-3 polypeptide identified according to the method of any one of claims 21 to 54.

56. The polypeptide of claim 55, wherein the polypeptide specifically binds a human LAG-3 polypeptide within amino acids 175 to 230 of the human LAG-3 polypeptide set forth in SEQ ID NO: 1.

57. The polypeptide of claim 55 or claim 56, wherein the polypeptide is an antibody.

58. A monoclonal antibody comprising the six CDRs of the antibody of claim 57.

59. The monoclonal antibody of claim 20 or claim 58, wherein the monoclonal antibody is an IgG.

60. The monoclonal antibody of claim 20 or claim 58, wherein the monoclonal antibody is selected from the group consisting of: a Fab, a F(ab′)₂, and a F(ab′).

61. The monoclonal antibody of claim 20 or claim 58, wherein the monoclonal antibody is a single chain antibody, optionally wherein the single chain antibody is an scFv.

62. The monoclonal antibody of any one of claims 20 or 58 to 61, wherein the monoclonal antibody is a bispecific antibody or a multi-specific antibody comprising a first antigen-binding domain that specifically binds domain 2 of a human LAG-3 polypeptide and a second antigen-binding domain.

63. The monoclonal antibody of claim 62, wherein the second antigen-binding domain specifically binds domain 2 of a human LAG-3 polypeptide, and wherein the epitope of domain 2 bound by the second antigen-binding domain is different from the epitope of domain 2 bound by the first antigen-binding domain.

64. The monoclonal antibody of claim 62, wherein the second antigen-binding domain specifically binds a domain of the human LAG-3 polypeptide other than domain 2.

65. The monoclonal antibody of claim 62, wherein the second antigen-binding domain specifically binds an antigen other than the human LAG-3 polypeptide.

66. The monoclonal antibody of any one of claims 20 or 58 to 65, wherein the monoclonal antibody is a pH-selective antibody, a protease-activated antibody, or a pH-selective and protease-activated antibody.

67. A method of identifying an agent as a candidate human LAG-3 dimerization disrupting agent, the method comprising:
conducting an in silico screen to identify an agent that interacts with the domain 2 dimerization interface of human LAG-3, wherein an agent that interacts with the domain 2 dimerization interface of human LAG-3 is a candidate human LAG-3 dimerization disrupting agent.

68. The method according to claim 67, wherein the conducting comprises conducting an in silico screen to identify an agent that interacts with an amino acid of the domain 2 dimerization interface of human LAG-3 selected from the group consisting of: A181, S182, W184, I186, N188, R196, E215, H220, A222, E223, F225, F227, P229, Q230, and any combination thereof, wherein numbering is according to SEQ ID NO:1.

69. The method according to claim 68, wherein the conducting comprises conducting an in silico screen to identify an agent that interacts with an amino acid of the domain 2 dimerization interface of human LAG-3 selected from the group consisting of: I186, E215, H220, E223, and any combination thereof.

70. The method according to claim 69, wherein the conducting comprises conducting an in silico screen to identify an agent that interacts with amino acid I186 of the domain 2 dimerization interface of human LAG-3.

71. The method according to claim 70, wherein the conducting comprises conducting an in silico screen to identify an agent that interferes with interaction between amino acid I186 of the domain 2 dimerization interface of human LAG-3 and one or each of amino acids A222 and F225 in the opposite monomer of a human LAG-3 dimer, wherein numbering is according to SEQ ID NO:1.

72. The method according to claim 69, wherein the conducting comprises conducting an in silico screen to identify an agent that interacts with amino acid E215 of the domain 2 dimerization interface of human LAG-3.

73. The method according to claim 74, wherein the conducting comprises conducting an in silico screen to identify an agent that interferes with interaction between amino acid E215 of the domain 2 dimerization interface of human LAG-3 and one or more amino acids in the opposite monomer of a human LAG-3 dimer.

74. The method according to claim 69, wherein the conducting comprises conducting an in silico screen to identify an agent that interacts with amino acid H220 of the domain 2 dimerization interface of human LAG-3.

75. The method according to claim 74, wherein the conducting comprises conducting an in silico screen to identify an agent that interferes with interaction between amino acid H220 of the domain 2 dimerization interface of human LAG-3 and one or each of amino acids W184 and I186 in the opposite monomer of a human LAG-3 dimer, wherein numbering is according to SEQ ID NO:1.

76. The method according to claim 69, wherein the conducting comprises conducting an in silico screen to identify an agent that interacts with amino acid E223 of the domain 2 dimerization interface of human LAG-3.

77. The method according to claim 76, wherein the conducting comprises conducting an in silico screen to identify an agent that interferes with interaction between amino acid E223 of the domain 2 dimerization interface of human LAG-3 and one, two or each of amino acids N188 glycan, E223 and F225 in the opposite monomer of a human LAG-3 dimer, wherein numbering is according to SEQ ID NO:1.

78. The method according to any one of claims 67 to 77, wherein the agent is a small molecule.

79. The method according to any one of claims 67 to 77, wherein the agent is a polypeptide.

80. The method according to any one of claims 67 to 77, wherein the agent is an antibody.

81. The method according to any one of claims 67 to 80, wherein the method further comprises screening the candidate human LAG-3 dimerization disrupting agent for the ability to disrupt human LAG-3 dimerization.

82. The method according to claim 81, further comprising identifying a candidate human LAG-3 dimerization disrupting agent that disrupts LAG-3 dimerization as a LAG-3 dimerization disrupting agent.

83. A pharmaceutical composition, comprising:
the monoclonal antibody of claims 20 or 58 to 66; and
a pharmaceutically acceptable carrier.

84. A pharmaceutical composition, comprising:
the polypeptide according to any one of claims 55 to 66; and
a pharmaceutically acceptable carrier.

85. A pharmaceutical composition, comprising:
a LAG-3 dimerization disrupting agent identified according to the method of claim 82; and
a pharmaceutically acceptable carrier.

86. A method comprising administering an effective amount of the pharmaceutical composition of any one of claims 83 to 85 to an individual in need thereof.

87. A method of administering an anti-LAG-3 immunotherapy to an individual in need thereof, the method comprising administering to the individual an effective amount of the pharmaceutical composition of any one of claims 83 to 85.

88. The method according to claim 87, wherein the anti-LAG-3 immunotherapy is administered as a monotherapy.

89. The method according to claim 87, wherein the anti-LAG-3 immunotherapy does not comprise administering an agent that targets an immune checkpoint molecule other than LAG-3 to the individual.

90. The method according to claim 87, wherein the anti-LAG-3 immunotherapy does not comprise administering an anti-PD1 agent to the individual.

91. The method according to any one of claims 87 to 90, wherein the individual has cancer, and wherein the anti-LAG-3 immunotherapy is effective in treating the cancer.

92. The method according to claim 91, wherein the cancer comprises a solid tumor.

93. The method according to claim 92, wherein the cancer comprises a carcinoma, lymphoma, blastoma, or sarcoma, optionally wherein the cancer comprises non-small cell lung cancer (NSCLC), head and neck squamous cell carcinoma (HNSCC), renal cell carcinoma (RCC), breast cancer, triple-negative breast cancer (TNBC), or melanoma.

94. The method according to claim 91, wherein the cancer is a hematologic malignancy.

95. The method according to claim 94, wherein the hematologic malignancy is a leukemia, a lymphoma, or multiple myeloma.
